(19) United States
(12) Patent Application Publication
(10) Pub. No.: US 2020/0197461 A1
(43) Pub. Date: Jun. 25, 2020

(54) NOVEL CANNABIS SATIVA LINES AND EXTRACTS WITH ANTI-CANCER PROPERTIES

(71) Applicants: Pathway RX Inc., Lethbridge (CA); Inplanta Biotechnology Inc., Lethbridge (CA)

(72) Inventors: Olga Kovalchuk, Lethbridge (CA); Dongping Li, Lethbridge (CA); Dwight Darryl Hudson, Raymond (CA); Igor Kovalchuk, Lethbridge (CA)

(21) Appl. No.: 16/711,647

(22) Filed: Dec. 12, 2019

Related U.S. Application Data
(60) Provisional application No. 62/781,631, filed on Dec. 19, 2018.

(51) Int. Cl. A61K 36/185

(52) U.S. Cl. CPC A61K 36/185

(57) ABSTRACT

The present invention provides new unique Cannabis lines, extracts and methods for their use in anti-cancer therapies and modalities. The method includes generation of unique lines, whole plant extract preparation, treating cancer cells and normal cells with extracts in amounts sufficient to kill cancer cells while sparing normal ones. The modulation of cell proliferation, growth and death results in efficient elimination of cancer cells.

### FIELD OF THE INVENTION

The present invention relates generally to products and methods for treating cancer, and more specifically to methods and products for treating cancer from Cannabis and hemp plants.

### BACKGROUND OF THE INVENTION

Malignancies encompass a wide array of diseases where abnormal cells divide and grow in an uncontrolled fashion, invade nearby tissues, and further spread to distant tissues and organs through the blood and lymphatic systems. Malignant tumors can arise from different cell types. Malignancies of epithelial origin are generally referred to as cancers. Central nervous system malignancies involve the brain and spinal cord. Soft tissue tumors, known as sarcomas, arise from mesenchymal cells of the connective and support tissues, such as from cartilage, bone, and muscle.

Leukemia is a blood malignancy originating from the bone marrow, and lymphoma and multiple myeloma are malignant immune system tumors. In common, as well as research terms, malignant tumors are often referred to as cancers.

Cancer is not a single disease, but rather a broad term applied to a variety of diseases involving the loss of cellular differentiation and abnormal growth of cells of epithelial origin, which have the potential to spread and invade other organs and body parts. Cancer cells are different from normal cells and their phenotypes exhibit three notable characteristics: (i) immortalization or indefinite proliferative life span; (ii) lack of response to conventional regulators of cell growth; and (iii) metastasis, the capacity to leave a tumor and invade distal tissues and organs.

Despite the efforts of the research and healthcare communities, cancer remains a growing health concern. It continues to be one of the leading causes of death worldwide, claiming 8.8 million lives in 2015 alone. Currently, 1 out of every 6 deaths in the world is attributable to cancer.

The number of cancer cases continues to rise. According to the World Health Organization, there were approximately 14 million new cancer cases in 2012, and the global incidence of cancer is projected to increase by around 70% in the upcoming decades. According to the estimates of the American Cancer Society, by 2030, the world will see up to 21.7 million new cancer cases and 13 million cancer deaths. Undeniably, the global economic impact of cancer is a growing burden; the economic cost of cancer in 2010 alone was estimated at approximately 1.16 trillion US dollars.

According to predictions of the Canadian Cancer Society, 1 in 2 Canadians will develop cancer in their lifetimes, and about 1 in 4 Canadians will succumb to it. It is estimated that 206,200 new cancer cases and 80,800 cancer deaths will occur 2017. Canadian males have a 45% lifetime probability and females have a 42% lifetime probability of developing cancer.

Chemotherapy is one of the main types of cancer treatment. It is based on the application of drugs that eradicate cancer cells (Hassan, Ansari et al. 2010). In most cases, chemotherapy is systemic, administered intravenously through the bloodstream to cells all over the body, thus affecting not only cancer cells, but also healthy cells.

Chemotherapy treatments can be classified as (i) neoadjuvant (pre-operative) chemotherapy, which is applied to reduce the size of a tumor prior to radiation therapy or surgical resection (Thompson and Moulder-Thompson 2012); (ii) adjuvant therapy, in which chemotherapeutic agents are applied to eradicate residual cancer after other treatments (Anampa, Makower et al. 2015); and (iii) combination therapy, in which chemotherapeutic agents are applied to eradicate cancer either by itself or in conjunction with other therapies (Hassan, Ansari et al. 2010). Depending on the patient's cancer type, stage, grade, underlying conditions, and other treatments used, chemotherapy can be applied to either cure or control cancer. It can also be used in a palliative setting to reduce tumor burden, lighten tumor pressure on organs, and thereby ease pain (DeVita, Hellman et al. 2005).

Depending on the types of agents used, chemotherapy is classified as cytotoxic (non-targeted) or targeted. Chemotherapy agents are designed to target rapidly dividing cells, and affect both tumor cells and healthy cells. As a result, they can cause numerous debilitating side effects.

Furthermore, long-term exposure to chemotherapy agents may eventually lead to acquisition of drug resistance, which is often the cause of treatment failure. Therefore, new drug discovery is always drawing much attention for both pharmacologists and medical doctors. There is a constant and ever-growing need for new and effective anti-cancer agents with reduced or minimized side effects.

U.S. Pat. No. 9,095,554 B2 discloses compositions and methods for the breeding, production, processing and use of specialty Cannabis.

There thus remains an unmet urgent and increasing need to provide effective non-toxic cancer therapies, methods for their manufacture and methods of cancer treatment.

### SUMMARY OF THE INVENTION

It is an object of some aspects of the present invention to provide effective non-toxic cancer therapies, methods for their manufacture and methods of cancer treatment.

In some embodiments of the present invention, improved methods and apparatus are provided for effective non-toxic cancer therapies, methods for their manufacture and methods of cancer treatment.

It is an object of some aspects of the present invention to provide compositions for improving wellness in a human or mammalian organism.

It is another object of some aspects of the present invention to provide compositions for preventing or treating diseases or disorders in a human or mammalian organism.

The compositions and dosage forms of the present invention are useful in promoting health and preventing or treating a large number of disorders in human patients and other mammalian subjects.

In additional embodiments of the present invention, compositions and methods are provided for treating and/or preventing proliferative disorders.

In additional embodiments of the present invention, compositions and methods are provided for treating and/or preventing cancer.

In additional embodiments of the present invention, compositions and methods are provided for treating and/or preventing skin disorders.

The present invention is directed to compositions and methods for treating disorders, in general, and more particularly, proliferative disorders and diseases. The compositions of the present invention may be used for improving wellness of a human or mammalian subject. Additionally, the compositions of the present invention may be used to treat any disorder or ailment in a human patient or mammalian subject. Furthermore, the compositions of the present invention may conveniently be used in conjunction with a drug to treat any disorder or ailment in a human patient or mammalian subject.

In other some embodiments of the present invention, a method is described for providing effective non-toxic cancer therapies, derived from Cannabis sativa (marijuana and/or hemp) plants, from now on referred to as “Cannabis”.

The present invention further provides new unique Cannabis lines, extracts, dried powders from the extracts, compositions comprising the powders or parts thereof, compounds derived therefrom, pharmaceutical compositions comprising the compound(s) and methods for their use in anti-cancer therapies and modalities. The method includes generation of unique lines, whole plant extract preparation, treating cancer cells and normal cells with extracts in amount sufficient to kill cancer cells while sparing normal (non-proliferative) ones. The modulation of cell proliferation, growth and death results in efficient elimination of cancer cells in response to the anti-cancer therapies and modalities of the present invention.

Cannabis has been suggested to harbor anti-cancer potential, however it has only gained much attention in the last two decades. While the most prevalent cannabinoids-Δ9-THC (tetrahydrocannabinol), cannabidiol (CBD), and cannabinol (CBN) were shown to affect cellular growth and proliferation, it is well-known that whole plant extracts have a milieu of other active molecules (terpenes, etc). Some terpenes may also have cytotoxic properties, thus full extracts exhibit the so-called combined “entourage effects”.

The present invention provides new Cannabis sativa (marijuana and hemp) lines, hybrids and cultivars and combinations thereof, as well as extracts thereof and methods of using them as a means to reduce viability and/or kill cancer cells while sparing normal ones. The disclosure also provides methods of modulating cell cycle, cell growth and cell death through the application of extracts of novel Cannabis lines on cancer cell line models.

The present invention further provides new Cannabis sativa lines and extracts and method of using them as a means to reduce viability and/or kill cancer cells while sparing normal ones. The disclosure also provides methods of modulating cell cycle, cell growth and cell death through the application of extracts of novel Cannabis lines on cancer cell line models.

Accordingly, the disclosure provides a means for modulating gene expression by Cannabis and/or hemp extracts (e.g., in skin tissues after exposure to UV light, a known carcinogen) by providing amounts sufficient to modulate gene expression where modulation of gene expression results in suppression of cell growth.

Further disclosed are over a hundred freshly prepared extracts of Cannabis sativa lines and a plurality of identified extracts displaying very pronounced anti-cancer activity. These include, but are not limited to, lines numbered CD10, #4, #10, #18, #20, #24, #28, #30, #33, #40, #41 and #81.

According to some additional embodiments of the present invention, the use of luminal A breast cancer (MCF-7), triple negative breast cancer cell line (HCC1806), colon cancer (HT29), glioblastoma (A172) and normal fibroblast cell lines WI-38, BJ-5ta, and HMEC as a model systems, it was shown that new Cannabis line extracts strongly suppress breast cancer cell proliferation and cell cycle arrest in a dose-dependent manner.

In other embodiments of the present invention, extracts of new Cannabis lines were also highly effective against neuroblastoma (IMR 5 model), Ewing sarcoma (SK-N-MC model), atypical teratoid/rhabdoid tumor (BT-12, BT-16 models).

In other embodiments of the present invention, a potent anti-cancer activity of novel Cannabis line extracts was found to be effective against cancer with a differential/benefit effect on normal cells, and may present a novel and promising natural resource for anti-cancer treatment and drug discovery.

In other embodiments of the present invention, extracts of new Cannabis lines were also able to inhibit proliferation of doxorubicin-resistant cancer cells.

Yet, in other embodiments of the present invention, extracts of new Cannabis lines potentiated the effect of chemotherapy agents such as cisplatin.

The present invention is directed to methods and products for treating proliferative and other disorders in human patients.

In particular, there are described methods for preparing compositions, compounds, formulations and extracts for treating a proliferative disorder in a human patient.

There is thus provided according to some embodiments of the present invention, a composition, derived from at least one of hemp and Cannabis for treating a proliferative disorder in a human patient.

A use of a solvent extract from flowers or/and leaves of at least one of hemp and Cannabis, according to some embodiments of the present invention, is for the manufacture of a pharmaceutical composition for the treatment of a proliferative disorder selected from the group consisting of a myeloma, multiple myeloma, breast cancer, glioblastoma, neuroblastoma, colorectal cancer, a malignant tumor, and brain stem glioma, and other disorders in human patients.

Some embodiments of the present invention are directed to a method for treating a proliferative disorder in a human patient comprising administering to said patient a pharmaceutically effective amount of the Cannabis extract composition as described herein.

Additionally, some further embodiments of the present invention are directed to a method for treating a proliferative disorder in a human patient comprising administering to said patient the oral dosage form as described herein.

The liquid Cannabis extracts of the present invention, and/or dry powders therefrom, are suitable for oral administration, and appear to be well absorbed through the intestine by the blood and thus exhibit the potential to heal a wide range of cancerous organs and inflammatory conditions, such as, but not limited to those mentioned by Chattopadhyay et al. Current Science 87(1) July 2004, 44-53.

According to some embodiments of the present invention, the composition or formulation further comprises at least one solvent or hydrant. In some cases, the hydrant is water, such as double-distilled water. In some cases, it may be at least one organic solvent, such as alcohol.

According to some embodiments of the present invention, the at least one solvent or hydrant is present in the composition or formulation in a concentration of 1-99.9%, 10-90%, 15-80%, 20-70%, 25-50%, 30-40%, or 10-18% by weight percent.

The solvent or hydrant may further comprise a pH regulator, such as an acid or base. In some embodiments, the base comprises sodium hydroxide.

For topical applications, suitable products or compositions of the present invention may be in the form of ointments or salves, creams, emulsions, gels, foams, sprays or medicated dressings or bandages, which must be directly applied on the affected zone and may be kept in contact with the skin.

In one or more embodiments, the compositions further comprise up to 10% of water.

In one or more embodiments, the composition is substantially non-aqueous and/or substantially alcohol-free.

In another embodiment, the present invention provides a method for inhibiting a disease in a subject comprising administering a subject a composition of the invention.

In another embodiment, the present invention provides a method for inhibiting a proliferative disease in a subject comprising administering a subject a composition of the present invention.

In another embodiment, the present invention provides a method for inhibiting a disease in a subject comprising orally administering a product of the present invention to the subject.

In another embodiment, the composition of the present invention is in a chewable oral dosage form. In another embodiment, the chewable oral dosage form is a chewable tablet. In another embodiment, the chewable tablet of the invention is taken slowly by chewing or sucking in the mouth. In another embodiment, the chewable tablet of the invention enables the dried Cannabis extracts contained therein to be orally administered without drinking.

In one or more embodiments, the composition further comprises a therapeutically effective concentration of one or more active agents.

The composition of the present invention further contains a surface-active agent. Surface-active agents (also termed “surfactants”) include any agent linking oil and water in the composition, in the form of emulsion.

In an embodiment of the present invention, a composition of the present invention includes one or more additional components. Such additional components include but are not limited to anti-static agents, buffering agents, bulking agents, chelating agents, cleansers, colorants, conditioners, diluents, dyes, emollients, fragrances, humectants, permeation enhancers, pH-adjusting agents, preservatives, protectants, skin penetration enhancers, softeners, solubilizers, sunscreens, sun blocking agents, sunless tanning agents, viscosity modifiers and vitamins. As is known to one skilled in the art, in some instances a specific additional component may have more than one activity, function or effect.

### EMBODIMENTS

1. A method for treating a mammalian disease, the method comprising:
   a) combining at least one marijuana or hemp cultivar and at least one other marijuana or hemp cultivar to form at least one hybrid line;
   b) extracting at least one compound from said at least one hybrid line; and
   c) administering said at least one compound in at least one of in vivo and in vitro to treat, slow or cure said mammalian disease.
2. A method according to embodiment 1, wherein said at least one hybrid line is selected from the group consisting of a marijuana/marijuana hybrid line, hemp/hemp hybrid line and hemp/marijuana hybrid line.
3. A method according to embodiment 2, wherein said at least one hybrid line is selected from the group consisting of hybrid number CD10, #4, #10, #18, #20, #24, #28, #30, #33, #40, #41 and #81.
4. A method according to embodiment 1, wherein said extracting step comprises extracting flowers or leaves or both of said at least one hybrid line.
5. A method according to embodiment 4, wherein said extracting step comprises extracting flowers or leaves or both in at least one organic solvent.
6. A method according to embodiment 5, wherein said extracting step is performed at a temperature in the range of 15-60° C. and at a pressure in a range of −0.5 to +1.5 bar.
7. A method according to embodiment 1, wherein said mammalian disease is a proliferative mammalian disease.
8. A method according to embodiment 8, wherein said proliferative mammalian disease is selected from the group consisting of cancer, breast cancer, colon cancer, glioblastoma, neuroblastoma, Ewing sarcoma, Atypical Teratoid Rhabdoid Tumor (ATRT), a cell-cycle deregulation disease, a metastatic disease and combinations thereof.
9. A method according to embodiment 1, wherein said at least one compound is provided in a concentration in a range of 0.0001-0.05 μg/μl, 0.001-0.05 μg/μl, 0.001-0.005 μg/μl, 0.003-0.03 μg/μl or 0.007-0.015 μg/μl.
10. A method according to embodiment 1, wherein said at least one compound is provided in a solvent extract and said solvent extract exhibits general anti-cancer effects, effective against a broad range of cancers.
11. A method according to embodiment 10, wherein said extract is effective against chemo-resistant breast cancer cells and is suitable to overcome chemo-resistance.
12. A method according to embodiment 10, wherein said extract potentiates effects of cytotoxic chemotherapy and is an effective and safe adjuvant modality.
13. A method according to embodiment 10, wherein said solvent extract is at least 2-20, 3-15, 4-12, 5-10 or 6-9 times as effective as at least one of THC (tetrahydrocannabinol) and CBD (cannabidiol), administered at the same concentration in treating said disease.
14. An organic extract of at least one hybrid line, said at least one hybrid line formed from combining at least one of:
   a) at least one marijuana or hemp cultivar; and
   b) at least one other marijuana or hemp cultivar,
      wherein said organic extract comprises at least one compound suitable for treating a mammalian disease.
15. An organic extract according to embodiment 14, wherein said mammalian disease is a proliferative mammalian disease, selected from the group consisting of cancer, breast cancer, neuroblastoma, Ewing sarcoma, Atypical Teratoid Rhabdoid Tumor (ATRT), a cell-cycle deregulation disease, a metastatic disease and combinations thereof.
16. An organic extract according to embodiment 15, wherein said extract is effective against chemo-resistant breast cancer cells and is suitable to overcome chemo-resistance.
17. An organic extract according to embodiment 15, wherein said extract potentiates effects of cytotoxic chemotherapy and is an effective and safe adjuvant modality.
18. An organic extract according to embodiment 15, wherein said organic extract is at least 2-20, 3-15, 4-12, 5-10 or 6-9 times as effective as at least one of THC and CBD, administered at the same concentration in treating said disease.
19. A combination therapy, isolated from an organic extract of at least one hybrid line, said at least one hybrid line formed from combining at least one of:
   a) at least one marijuana or hemp cultivar; and
   b) at least one other marijuana or hemp cultivar; and
      wherein said organic extract comprises a plurality of compounds suitable for treating a mammalian disease.
20. A combination therapy according to embodiment 19, wherein said mammalian disease is a proliferative mammalian disease, selected from the group consisting of cancer, breast cancer, colon cancer, glioblastoma, neuroblastoma, Ewing sarcoma, Atypical Teratoid Rhabdoid Tumor (ATRT), a cell-cycle deregulation disease, a metastatic disease and combinations thereof.
21. A line of Cannabis sativa formed by combining at least one marijuana or hemp cultivar and at least one other marijuana or hemp cultivar, said line to be deposited at publicly available culture collection under a specified designation number, designated herein CD10, #4, #10, #18, #20, #24, #28, #30, #33, #40, #41 or #81.

The present invention will be more fully understood from the following detailed description of the some embodiments thereof, taken together with the drawings.

### BRIEF DESCRIPTION OF THE DRAWINGS

The invention will now be described in connection with certain some embodiments with reference to the following illustrative figures so that it may be more fully understood.

With specific reference now to the figures in detail, it is stressed that the particulars shown are by way of example and for purposes of illustrative discussion of the some embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the context of this application, the word “Figure” is abbreviated to “Fig.”.

In the drawings:

FIGS. 1A-1L provide examples of high performance liquid chromatography (HPLC) profiles of tested lines, in accordance with some embodiments of the present invention;

FIG. 2 is a simplified pictorial illustration of a method for identifying solvent plant extracts having anti-cancer properties, in accordance with some embodiments of the present invention;

FIG. 3A shows a group of simplified graphical flow cytometer outputs of analysis of the cell cycle deregulation, induced by Cannabis extract (#3) in triple-negative breast squamous cell carcinoma HCC1806 breast cancer cells, in accordance with some embodiments of the present invention. “Untreated”—untreated cells; “DMSO”—DMSO treated cells; “24 h 0.007 ug/ul”—changes in cell cycle in response to 0.007 ug/ul 24 h after application of the extract #3; “48 h 0.007 ug/ul”—changes in cell cycle in response to 0.007 ug/ul 48 h after application of the extract #3;

FIG. 3B shows a group of simplified graphical flow cytometer outputs of analysis of the cell cycle deregulation, induced by Cannabis extract (#3) in triple-negative breast squamous cell carcinoma HCC1806 breast cancer cells, in accordance with some embodiments of the present invention; “24 h 0.015 ug/ul”—changes in cell cycle in response to 0.015 ug/ul 24 h after application of the extract #3; “48 h 0.015 ug/ul”—changes in cell cycle in response to 0.015 ug/ul 48 h after application of the extract #3; “24 h 0.03 ug/ul”—changes in cell cycle in response to 0.03 ug/ul 24 h after application of the extract #3; “48 h 0.03 ug/ul”—changes in cell cycle in response to 0.03 ug/ul 48 h after application of the extract #3;

FIG. 3C shows calculation of the percentage of cells in a specific cell cycle, based on the results shown in FIG. 3A-3B. Asterisks show significant change (p<0.05) compared to DMSO treatment. Panel on the right shows an example of the cell cycle graphical output with description of various cell cycle phases.

FIG. 3D shows a summary table of the cell cycle effects screening different lines' candidate extracts, in accordance with some embodiments of the present invention; “Bold underlined” compounds/line extracts cause cell cycle arrest at both low and high concentration, “italic” compounds/line extracts cause cell cycle arrest at low concentration, “Underlined” compounds/line extracts cause cell cycle arrest at high concentration. Bold underlined: 56 out of 212=26.4%. 56 extracts were subjected to further testing using MTT, a colorimetric assay for assessing cell metabolic activity, leading to identification of 23 extracts positive in MTT assay; of which top 12 were further analyzed as the most potent ones.

FIG. 4A shows graphical results of cell growth (arbitrary units of proliferation) in the presence of eleven candidate Cannabis active extracts (#4, #10, #18, #20, #24, #28, #30 and #40) on normal human lung fibroblast W138 cells, and

FIG. 4B shows graphical results of cell growth (arbitrary units of proliferation) in the presence of nine candidate active extracts on BJ-5ta normal cells, demonstrating no inhibitory growth effect of the eight Cannabis candidate extracts (#4, #10, #18, #20, #24, #28, #30 and #40) on normal cells, in accordance with some embodiments of the present invention;

FIG. 5A provides graphical results of effects of four Cannabis extracts (#4, #10, #28, and #40) on HCC1806 breast cancer cell proliferation (arbitrary units of proliferation), in accordance with some embodiments of the present invention;

FIG. 5B provides graphical results of effects of four Cannabis extracts (#4, #10, #28, and #40) on MCF7 breast cancer cell proliferation (arbitrary units of proliferation), in accordance with some embodiments of the present invention;

FIG. 6A provides graphical results of effects of Cannabis line #40 extract on suppression of neuroblastoma growth (shown in arbitrary units of proliferation); in accordance with some embodiments of the present invention;

FIG. 6B shows the effects of Cannabis line #40 extract does not affect normal fibroblasts, in accordance with some embodiments of the present invention;

FIG. 7 provides graphical results of effects of extracts of Cannabis lines #81 and #10—left-hand panels, containing between 1% THC and 10% CBD on cell proliferation (arbitrary units of proliferation). Extracts exhibit more potent anti-cancer effects as compared to pure cannabinoids THC and CBD (right-hand panels), in accordance with some embodiments of the present invention;

FIG. 8 shows graphical results of the effects of two different extracts Cannabis lines (#4 and #20) on the growth of cancer cells (breast cancer, upper left panel, neuroblastoma lower left panel, and breast cancer triple negative, upper right panel), but not on normal cells (lower right panel) (arbitrary units of proliferation), in accordance with some embodiments of the present invention; and

FIG. 9 shows an output table of modulation of gene expression in four different pathways in response to human skin tissues treatment with Cannabis line #4 after exposure to a known cancer causing agent (UV radiation), affecting pathways involved in cancer, apoptosis and metastasis. “KEGGID” shows the pathway ID (from KEGG pathway software), “Pvalue” shows statistical significance, “Count” shows the number of genes altered in the pathway, “Size” shows total number of genes associated with a given pathway, “Term” shows the name of the pathway. A p value of less than 0.05 is considered to be significant, in accordance with some embodiments of the present invention.

FIG. 10A shows images of MCF-7/DOX cells were treated with DOX (48 h, 1 μg/ml) in combination with Cannabis extract #10 at 0.007, 0.015, 0.025 and 0.05 μg/μl;

FIG. 10B—shows a cell count of MCF-7/DOX cells, which were treated with DOX (48 h, 1 μg/ml) in combination with Cannabis extract #10 at 0.007, 0.015, 0.025 and 0.05 μg/μl;

FIG. 10C—shows images of MCF-7/DOX cells, which were treated with DOX (48 h, 1 μg/ml) in combination with Cannabis extract #20 at 0.007, 0.015, 0.025 and 0.05 μg/μl;

FIG. 10D—cell count of MCF-7/DOX cells were treated with DOX (48 h, 1 μg/ml) in combination with Cannabis extract #20 at 0.007, 0.015, 0.025 and 0.05 μg/μl;

FIG. 11A—images of MCF-7/DOX cells were treated with DOX (48 h, 1 μg/ml) in combination with Cannabis extract #28 at 0.007, 0.015, 0.025 and 0.05 μg/μl,

FIG. 11B—cell count of MCF-7/DOX cells were treated with DOX (48 h, 1 μg/ml) in combination with Cannabis extract #28 at 0.007, 0.015, 0.025 and 0.05 μg/μl;

FIG. 11C—images of MCF-7/DOX cells were treated with DOX (48 h, 1 μg/ml) in combination with Cannabis extract #41 at 0.007, 0.015, 0.025 and 0.05 μg/μl;

FIG. 11D—cell count of MCF-7/DOX cells were treated with DOX (48 h, 1 μg/ml) in combination with Cannabis extract #41 at 0.007, 0.015, 0.025 and 0.05 μg/μl;

FIG. 12—images of MCF-7/DOX cells were treated with DOX (48 h, 1 μg/ml) in combination with Cannabis extract #4 at 0.007, 0.015, 0.025 and 0.05 μg/μl, in accordance with some embodiments of the present invention;

FIG. 13A-13D show dose-dependent effect of extracts #4, 18, #24 and #33 on the proliferation (shown in arbitrary units) of HT29 colon cancer cells, in accordance with some embodiments of the present invention;

FIGS. 14A-14F show treatment of colon cancer cells HT29 with extracts #18, #24 and #33 at a concentration of 0.015 μg/μl potentiates the effect of 5 and 10 μM cisplatin on cell proliferation (shown in arbitrary units), in accordance with some embodiments of the present invention;

FIGS. 15A-15D show the effect of treatment (measured in arbitrary units of proliferation) of neuroblastoma cells SK-N-MC with extracts #4, #10, #20 and #28 at a concentration of 0.007 (low) and 0.015 (high) μg/μl, in accordance with some embodiments of the present invention;

FIGS. 16A-16D show the effect of treatment (measured in arbitrary units of proliferation) of atypical teratoid/rhabdoid tumor cells BT-12 with extracts #4, #10, #20 and #28 at a concentration of 0.007 (low) and 0.015 (high) μg/μl, in accordance with some embodiments of the present invention;

FIGS. 17A-17D show the effect of treatment (measured in arbitrary units of proliferation) of atypical teratoid/rhabdoid tumor cells BT-16 with extracts #4, #10, #20 and #28 at a concentration of 0.007 (low) and 0.015 (high) μg/μl, in accordance with some embodiments of the present invention;

FIG. 18 shows the effect of treatment (measured in arbitrary units of proliferation) of breast cancer cell HCC1806 with CD10 extract in accordance with some embodiments of the present invention;

FIGS. 19A-19D show the effect of treatment (measured in arbitrary units of proliferation) of neuroblastoma cells IMR5 cells with extracts #4, #10, #18, #20, at a concentration of 0.007 (low) and 0.015 (high) μg/μl, in accordance with some embodiments of the present invention; and

FIGS. 20A-20C show the effect of treatment (measured in arbitrary units of proliferation) of neuroblastoma cells IMR5 cells with extracts #24, #30 and #40 at a concentration of 0.007 (low) and 0.015 (high) μg/μl, in accordance with some embodiments of the present invention.

In all the figures similar reference numerals identify similar parts.

### DETAILED DESCRIPTION OF THE EMBODIMENTS

In the detailed description, numerous specific details are set forth in order to provide a thorough understanding of the invention. However, it will be understood by those skilled in the art that these are specific embodiments and that the present invention may be practiced also in different ways that embody the characterizing features of the invention as described and claimed herein.

The present invention provides new unique Cannabis lines, extracts and methods for their use in anti-cancer therapies and modalities. The method includes generation of unique lines, whole plant extract preparation, treating cancer cells and normal cells with extracts in amounts sufficient to kill cancer cells while sparing normal ones. The modulation of cell proliferation, growth and death results in efficient elimination of cancer cells.

The present invention further provides methods of drug discovery. According to some embodiments, the method includes:
a) combining at least one marijuana cultivar and at least one hemp cultivar to form at least one hybrid line;
b) extracting at least one compound from said at least one hybrid line to form an extract; and
c) testing the extract in vitro to identify a biologically active extract.

The method further includes repeating steps a) to c) on a plurality of extracts to identify the most biologically active extracts.

The method further includes isolating active compounds or components from the biologically active extracts.

The method further comprises treating a patient with a disease or disorder with at least one of the active compounds, components or extracts to cure, alleviate or manage the disease or disorder.

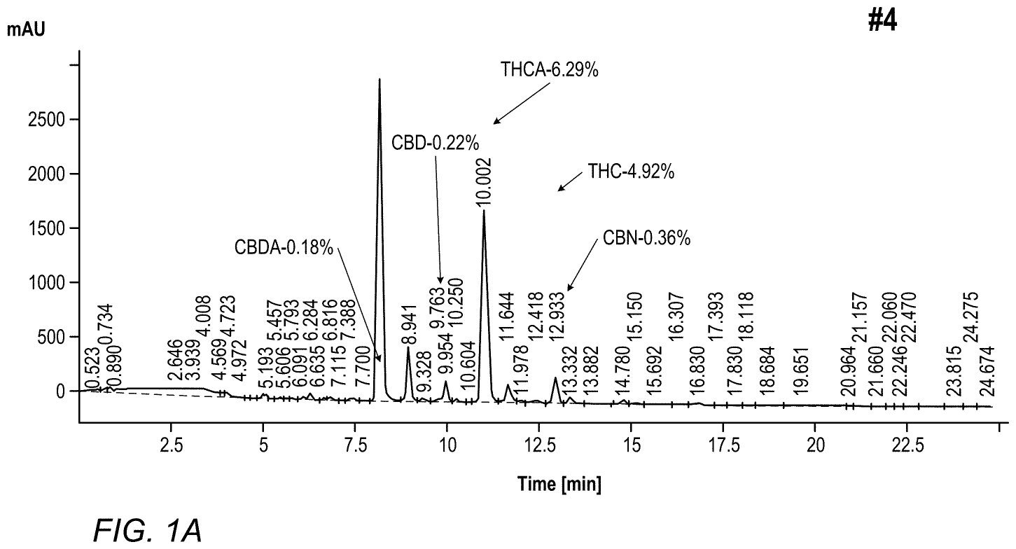

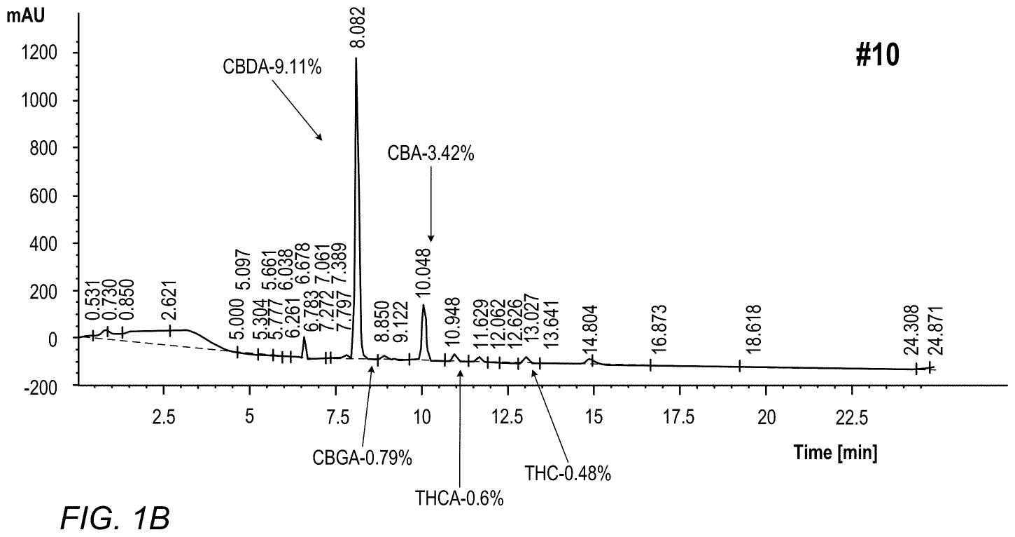

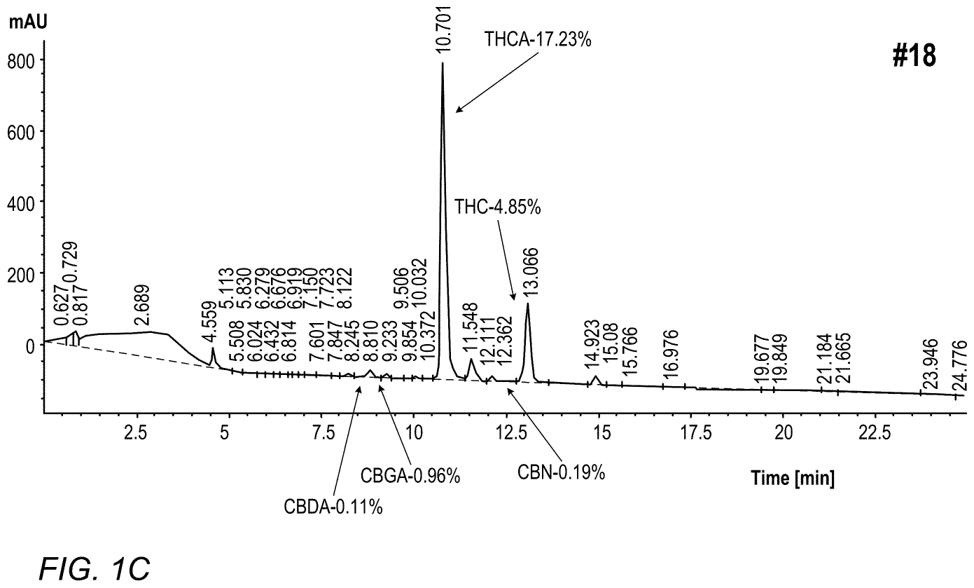

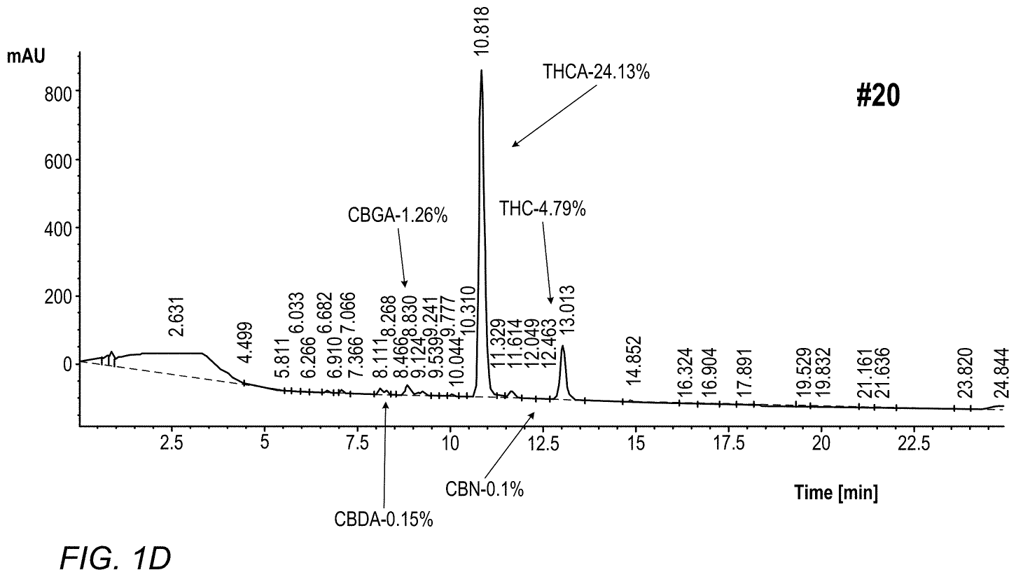

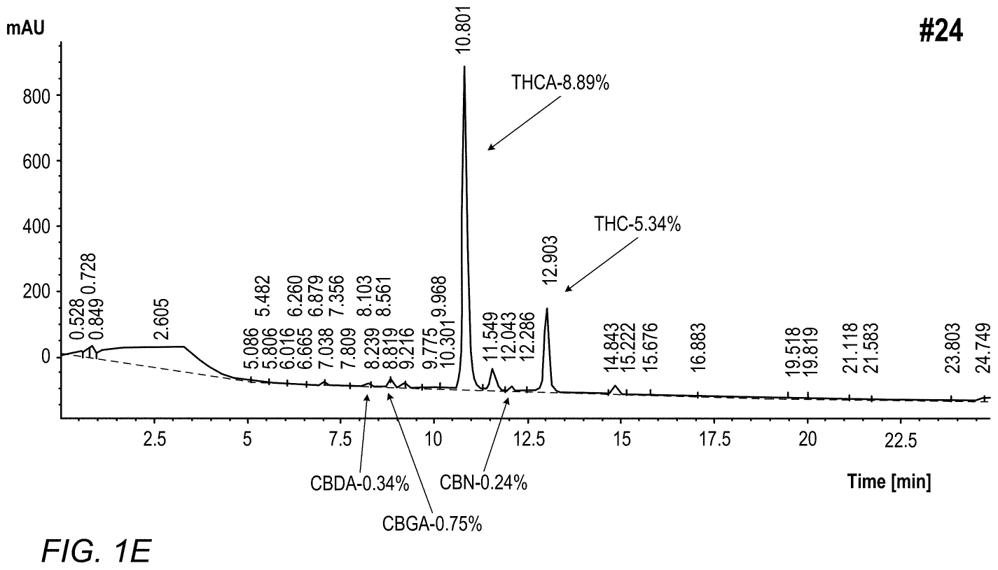

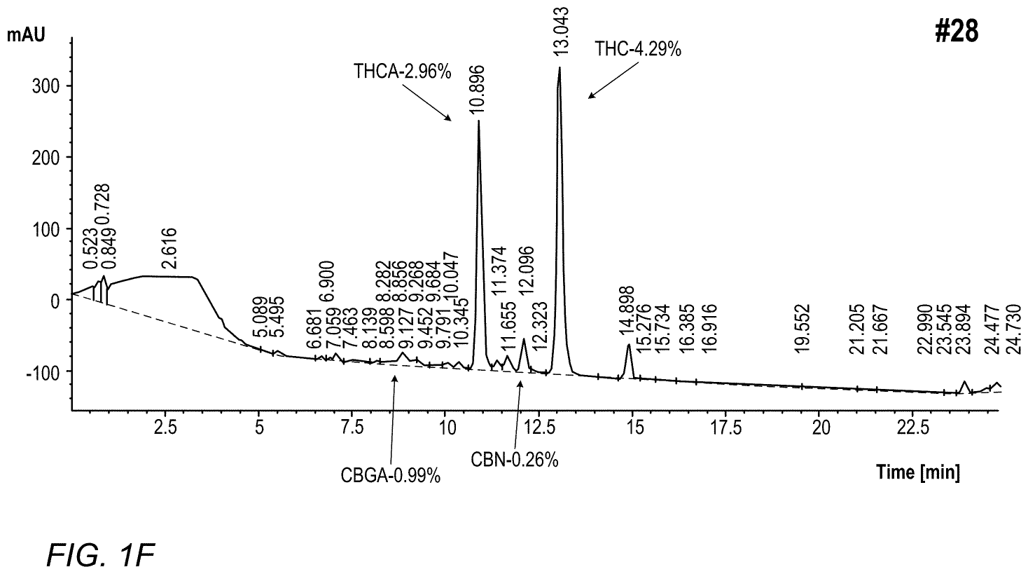

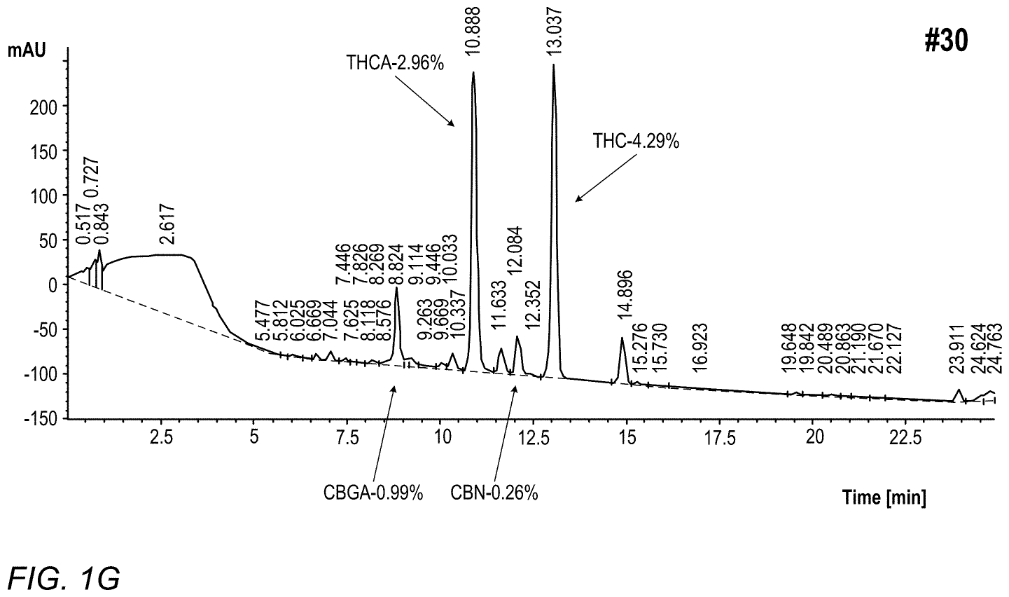

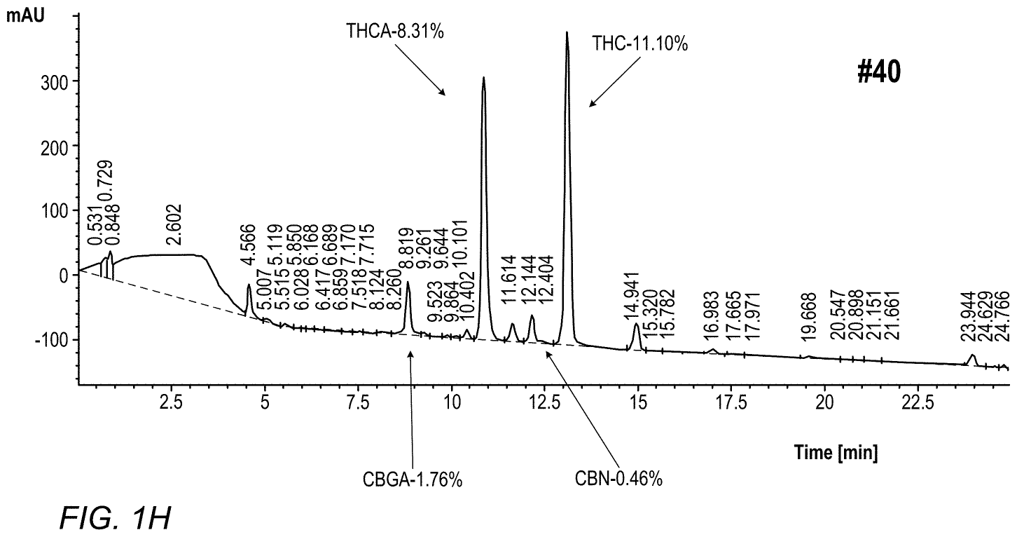

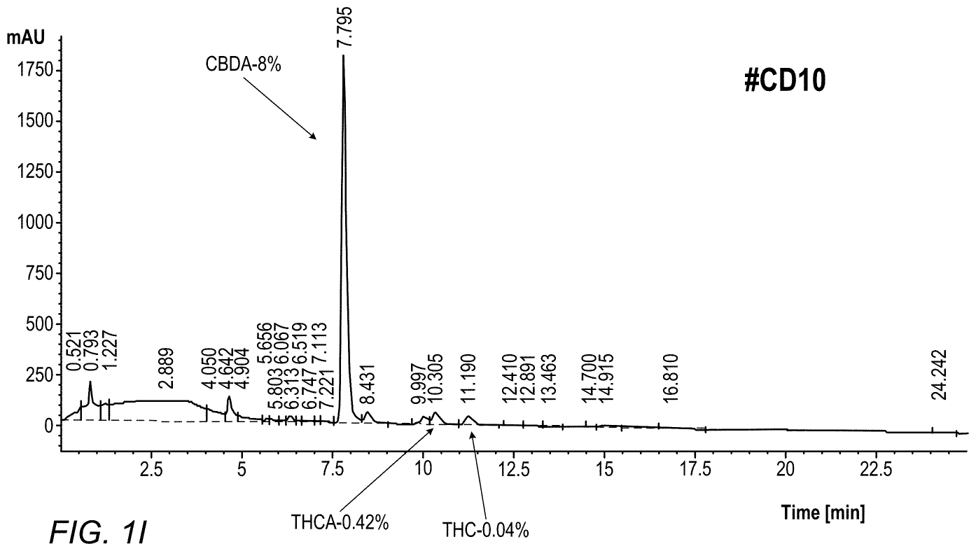

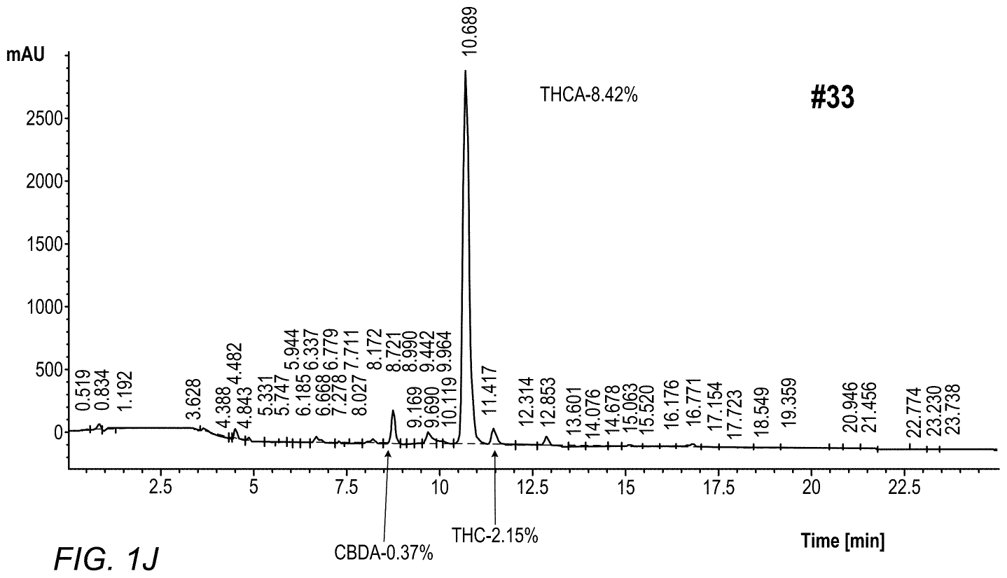

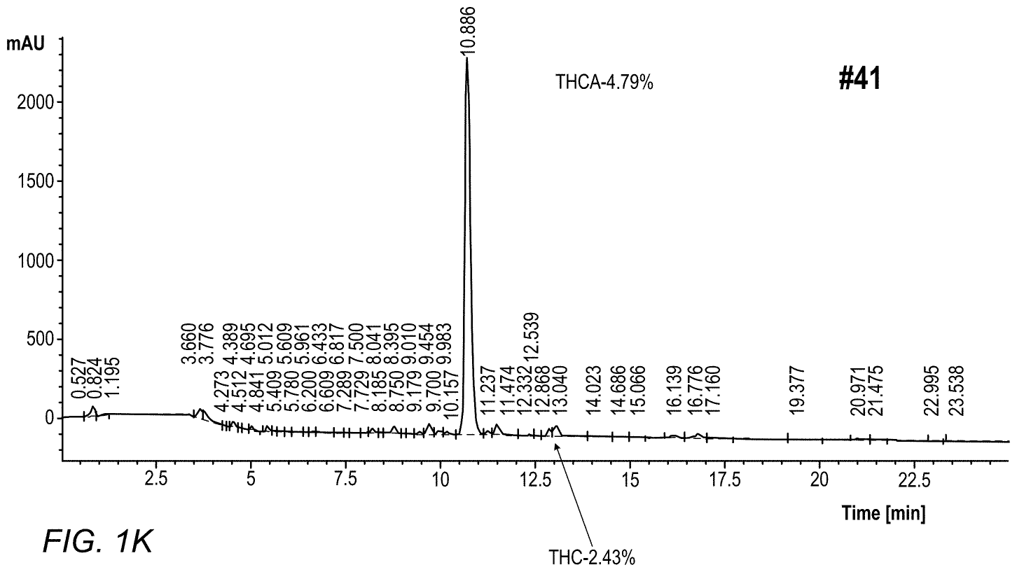

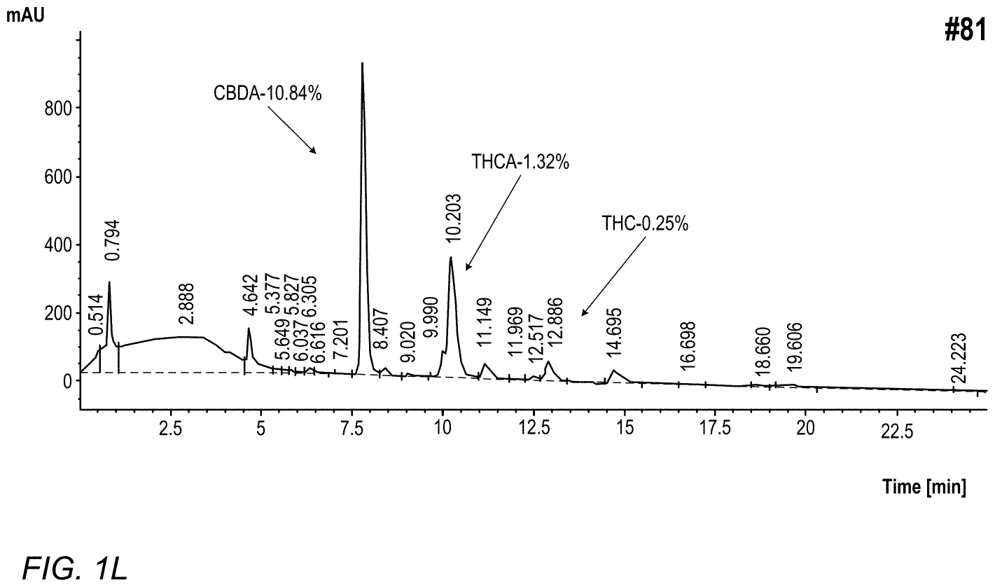

FIGS. 1A-1L provide high performance liquid chromatography (HPLC) profiles of tested lines, in accordance with some embodiments of the present invention.

FIG. 1A—chromatogram of #4 extract. Total THC equivalent 10.44% and CBD 0.38%.

FIG. 1B—chromatogram of #10 extract. Total THC equivalent 1.0% and CBD 11.41%.

FIG. 1C—chromatogram of #18 extract. Total THC equivalent 19.96% and CBD 0.1%.

FIG. 1D—chromatogram of #20 extract. Total THC equivalent 25.95% and CBD 0.13%.

FIG. 1E—chromatogram of #24 extract. Total THC equivalent 13.13% and CBD 0.64%.

FIG. 1F—chromatogram of #28 extract. Total THC equivalent 6.89%.

FIG. 1G—chromatogram of #30 extract. Total THC equivalent 9.93% and CBD 0.05%.

FIG. 1H—chromatogram of #40 extract. Total THC equivalent 18.38% and CBD 0.05%.

FIG. 1I—chromatogram of CD10 extract. Total THC equivalent 1.15% and CBD 14.9%.

FIG. 1J—chromatogram of #33 extract. Total THC equivalent 10.3% and CBD 0.35%.

FIG. 1K—chromatogram of #41 extract. Total THC equivalent 6.85% and CBD 0.1%.

FIG. 1L—Chromatogram of #81 extract. Total THC equivalent 1.35% and CBD 10.2%.

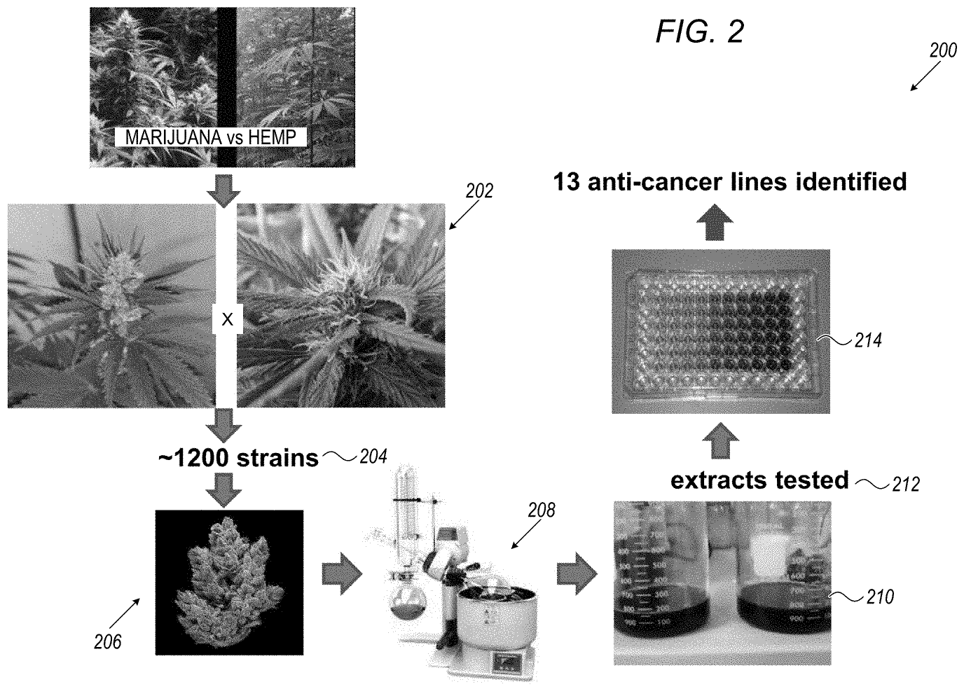

FIG. 2 is a simplified pictorial illustration of a method 200 for identifying solvent plant extracts having anti-cancer properties, in accordance with some embodiments of the present invention.

Step 202—a Cultivar Growing Step.

Around 250 unique marijuana and around 120 unique hemp cultivars were used to generate approximately 1,200 marijuana/marijuana, hemp/hemp and hemp/marijuana hybrids. Cultivars are typically grown in soil/vermiculite (2:1) mix. First, plants are grown under 16 h day, 8 h night for approximately 6 weeks when they were moved to another grow room and grown at 12 h day and 12 h night for another 6-8 weeks until they developed mature flowers. In both rooms, they were grown under the high pressure sodium (HPS) lights of ˜400 W/m2. Collected flowers were then tested for cannabinoids and terpenoids and those with most diversity in composition, or those that had highest amount of one or more cannabinoid or terpenoid or those that had the presence of unique terpenoids were used for breeding.

Step 204—a cultivar selection step. The progeny of these crosses was then grown and further tested for cannabinoids/terpenoids as well as for growth parameters, such as height, response to nutrients, responses to pathogens, amongst others. In some cases, these plants were then crossed again using siblings with similar traits (cannabinoids/terpenoids for example).

Step 205 a cultivar storage step. The seeds of these cultivars (resulting from crosses) are stored at +4° C. in the fridge in the locked cage.

Step 206 a cultivar growing step to generate flowers. Approximately 600 cultivars with the best parameters, such as diversity of cannabinoids and terpenoids, plant growth vigor (germination rate, mutation time, yield of flowers, nutrients response, response to pathogens, size of flowers) and other features such as distinct smell for example were germinated and approximately 400 extracts were made.

Step 208, an extraction step, such as organic solvent extraction. Most solvents can be used. In one experiment ethyl acetate was used. This should not be deemed as limiting. For extract preparation, 3 g of the powdered flower tissue were used in 100 ml of ethyl acetate in a 250 mL Erlenmeyer flask. The flasks were then wrapped with tin foil and shaken continuously (120 rpm) in an incubator at 21° C. overnight and in the dark. After overnight solvent extraction the extracts were filtered through cotton into a 100 ml round bottom flask. The extracts were concentrated to around 2-3 ml using a rotary vacuum evaporator. The extracts were then transferred to a tared 3 dram vial.

Step 210. The leftover solvent was evaporated to dryness in an oven overnight at 50° C. to eliminate the solvent completely. Mass of each extract was recorded, and the extracts were stored at −20° C. The stocks were prepared weighing a 3-6 mg of crude extract into a micro centrifuge tube. The crude extract was dissolved in DMSO (Dimethyl sulfoxide anhydrous) to reach 60 mg/mL final concentration and stored at −20° C. Around 400 solvent-based crude extracts of flowers were thus generated.

In an extract biological assay step 212, many of the selected extracts were tested as follows. Appropriate cell culture media (for example RPMI+10% FBS or EMEM+10% FBS) was used to dilute the 60 mg/mL stock. Appropriate amounts of stock extract were added to the cell culture media, mixed thoroughly to ensure they are in solution and filtered through a 0.22 um syringe filter. These filtrates were ready to be applied to cells and tested. For example, to achieve the concentration of 0.007 mg/ml, 2.45 μl of stock extract (60 mg/ml) was added to 21 ml of medium.

In an extract biological assay step 212, extracts were tested using breast cancer, neuroblastoma, Ewing sarcoma models and normal cells, and several best performing extracts were identified. First test included the analysis of cell cycle. Out of 400 extracts prepared, 212 were tested and 56 showed positive for cell cycle arrest.

All these 56 extracts were subjected to further testing of proliferation using MTT, a colorimetric assay for assessing cell metabolic activity, leading to identification of 23 extracts positive in MTT assay and exhibiting no effect on normal cells; of which top 12 were further analyzed as the most potent ones.

These were found to be, inter alia, CD10, #4, #10, #18, #20, #24, #28, #30, #33, #40, #41 and #81.

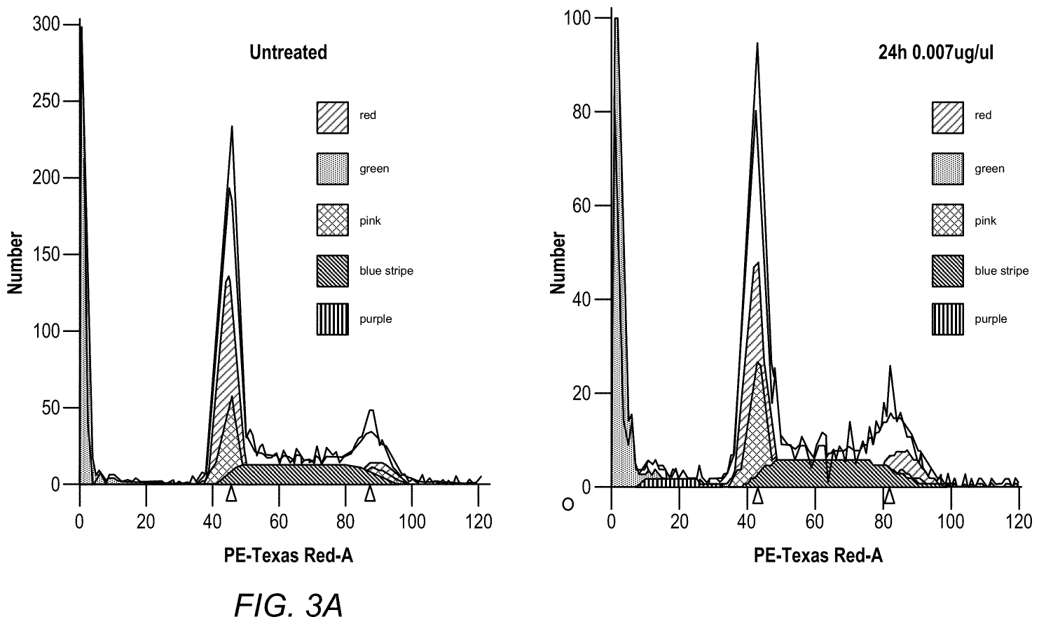

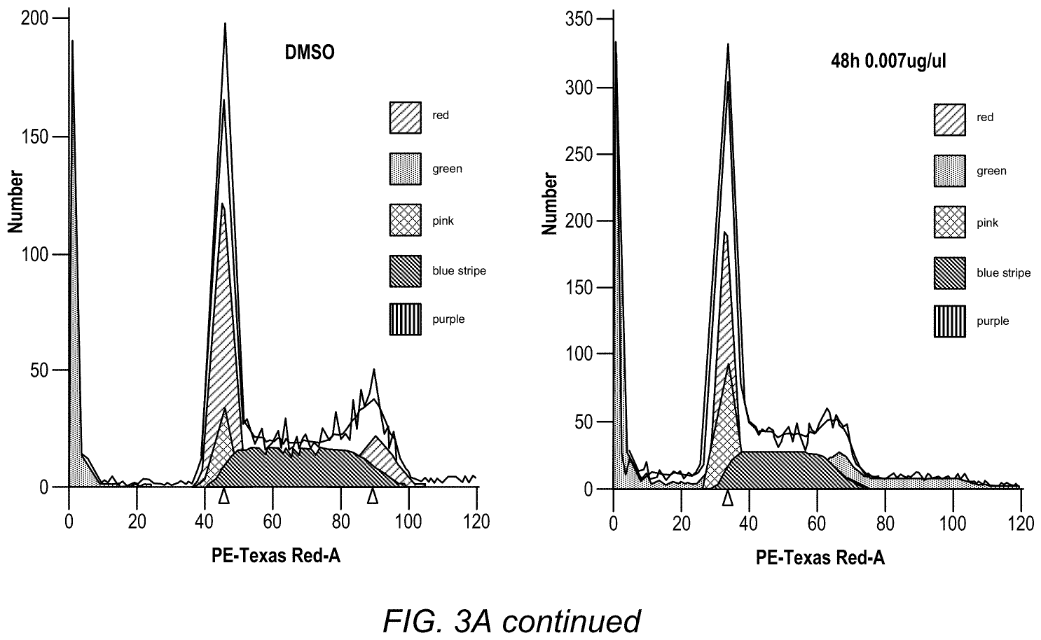

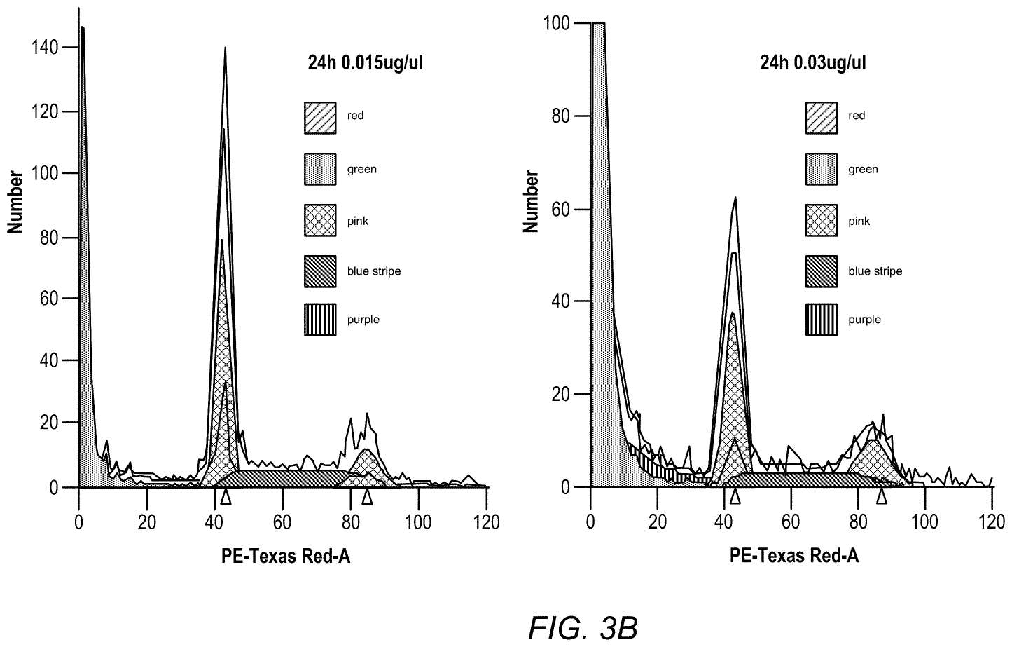

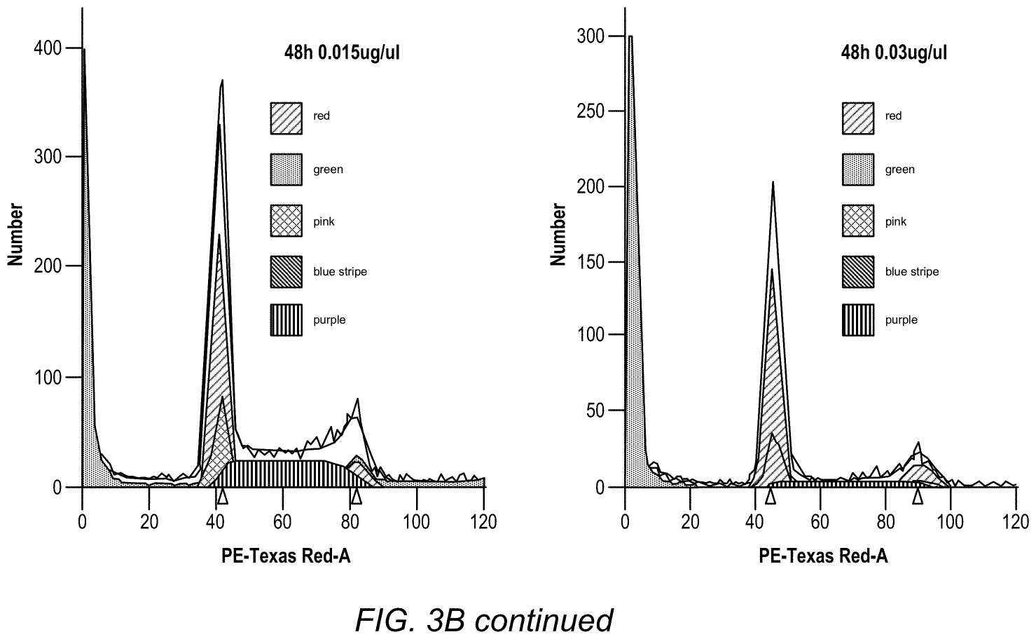

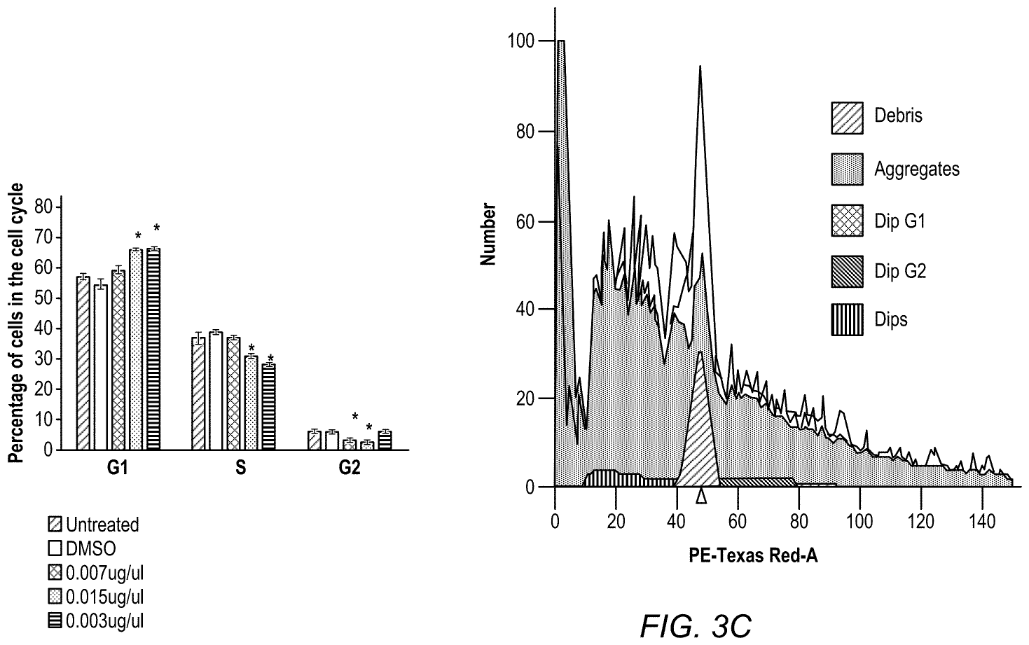

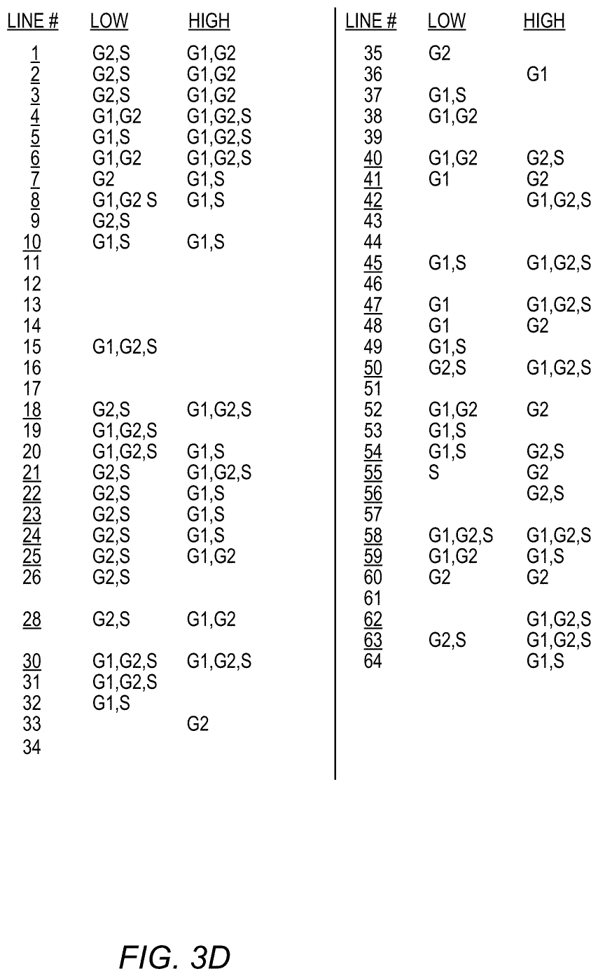

FIG. 3A-B show groups of simplified graphical flow cytometer outputs of analysis of the cell cycle deregulation, induced by Cannabis extract (#3) in triple-negative breast squamous cell carcinoma HCC1806 breast cancer cells, in accordance with some embodiments of the present invention.

FIG. 3A shows a group of simplified graphical flow cytometer outputs of analysis of the cell cycle deregulation, induced by Cannabis extract (#3) in triple-negative breast squamous cell carcinoma HCC1806 breast cancer cells, in accordance with some embodiments of the present invention. “Untreated”—untreated cells; “DMSO”—DMSO treated cells; “24 h 0.007 ug/ul”—changes in cell cycle in response to 0.007 ug/ul 24 h after application of the extract #3; “48 h 0.007 ug/ul”—changes in cell cycle in response to 0.007 ug/ul 48 h after application of the extract #3;

FIG. 3B shows a group of simplified graphical flow cytometer outputs of analysis of the cell cycle deregulation, induced by Cannabis extract (#3) in triple-negative breast squamous cell carcinoma HCC1806 breast cancer cells, in accordance with some embodiments of the present invention; “24 h 0.015 ug/ul”—changes in cell cycle in response to 0.015 ug/ul 24 h after application of the extract #3; “48 h 0.015 ug/ul”—changes in cell cycle in response to 0.015 ug/ul 48 h after application of the extract #3; “24 h 0.03 ug/ul”—changes in cell cycle in response to 0.03 ug/ul 24 h after application of the extract #3; “48 h 0.03 ug/ul”—changes in cell cycle in response to 0.03 ug/ul 48 h after application of the extract #3;

The eukaryotic cell cycle consists of four main stages:

G1, in which a cell is metabolically active and grows continuously;

S phase, during which DNA replication takes place;

G2, in which cell growth continues and the cell synthesizes various proteins in preparation for division; and

M (mitosis) phase, during which the duplicated chromosomes (known as the sister chromatids) separate into two daughter nuclei, and the cell divides into two daughter cells, each with a full copy of DNA.

FIG. 3C shows calculation of the percentage of cells in a specific cell cycle, based on the results shown in FIG. 3A-3B. Asterisks show significant change (p<0.05) compared to DMSO treatment. Panel on the right shows an example of the cell cycle graphical output with description of various cell cycle phases.

FIG. 3D shows a summary table of the cell cycle effects screening different lines' candidate extracts, in accordance with some embodiments of the present invention;

“Bold underlined” compounds/line extracts cause cell cycle arrest at both low and high concentration, “italic” compounds/line extracts cause cell cycle arrest at low concentration, “Underlined” compounds/line extracts cause cell cycle arrest at high concentration. Bold underlined: 56 out of 212=26.4%. 56 extracts were subjected to further testing using MTT, a colorimetric assay for assessing cell metabolic activity, leading to identification of 23 extracts positive in MTT assay; of which top 12 were further analyzed.

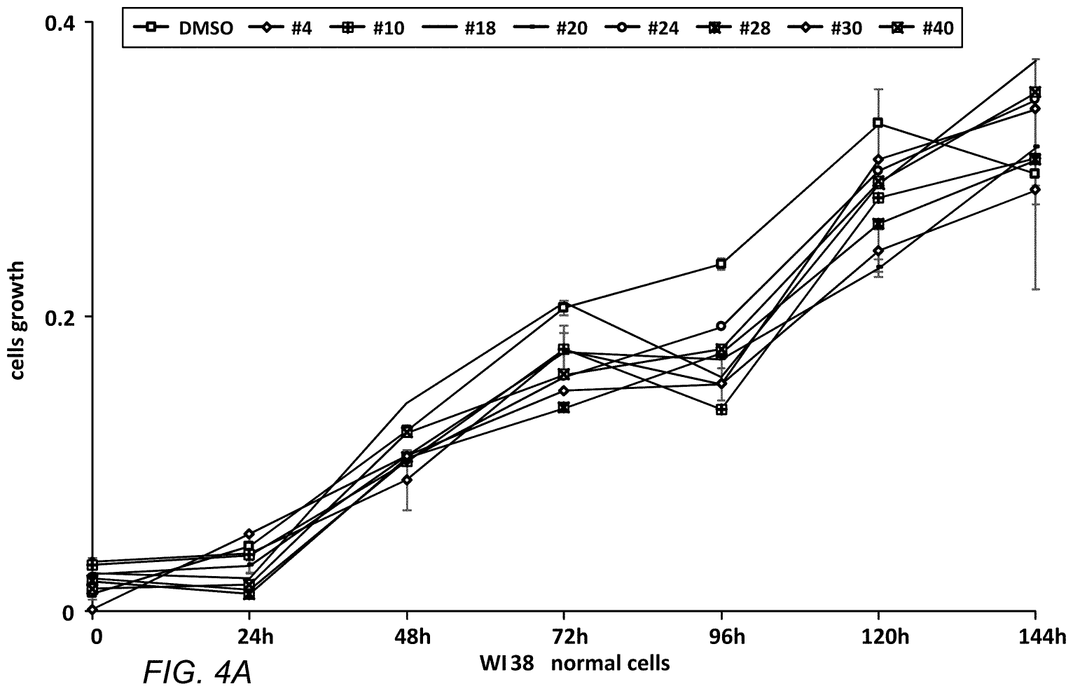

FIG. 4A shows graphical results of cell growth (arbitrary units of proliferation) in the presence of eight candidate Cannabis active extracts (4, #10, #18, #20, #24, #28, #30 and #40) on normal human lung fibroblast W138 cells, and FIG. 4B shows graphical results of cell growth in the presence of eight candidate active extracts on BJ-5ta normal cells, demonstrating no inhibitory growth effect of the nine Cannabis candidate extracts (#4, #10, #18, #20, #24, #28, #30 and #40) on normal cells, in accordance with some embodiments of the present invention.

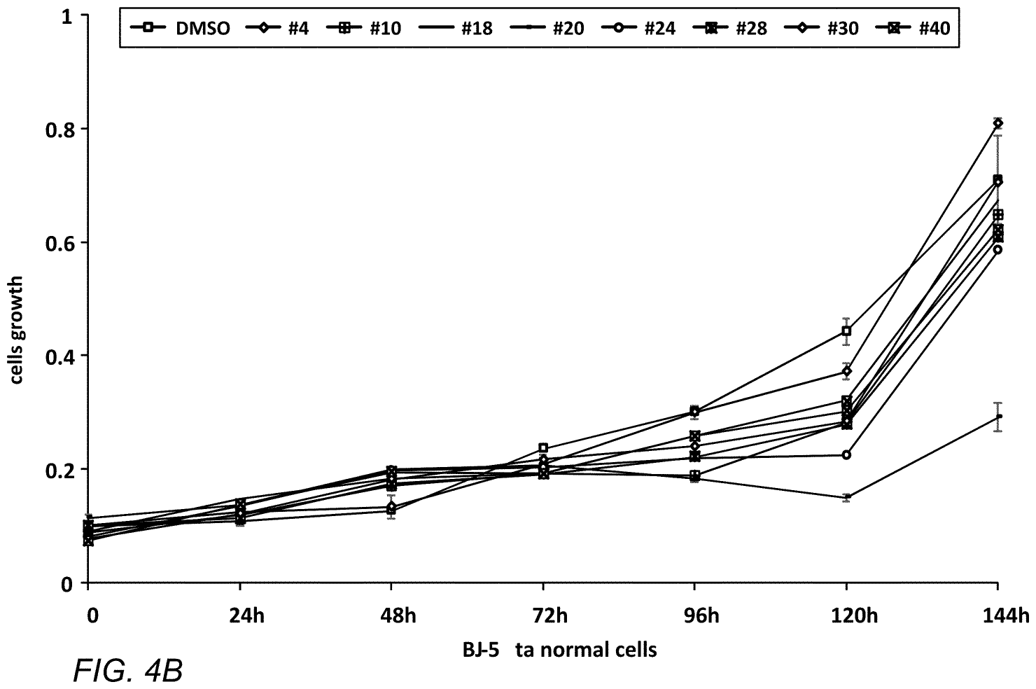

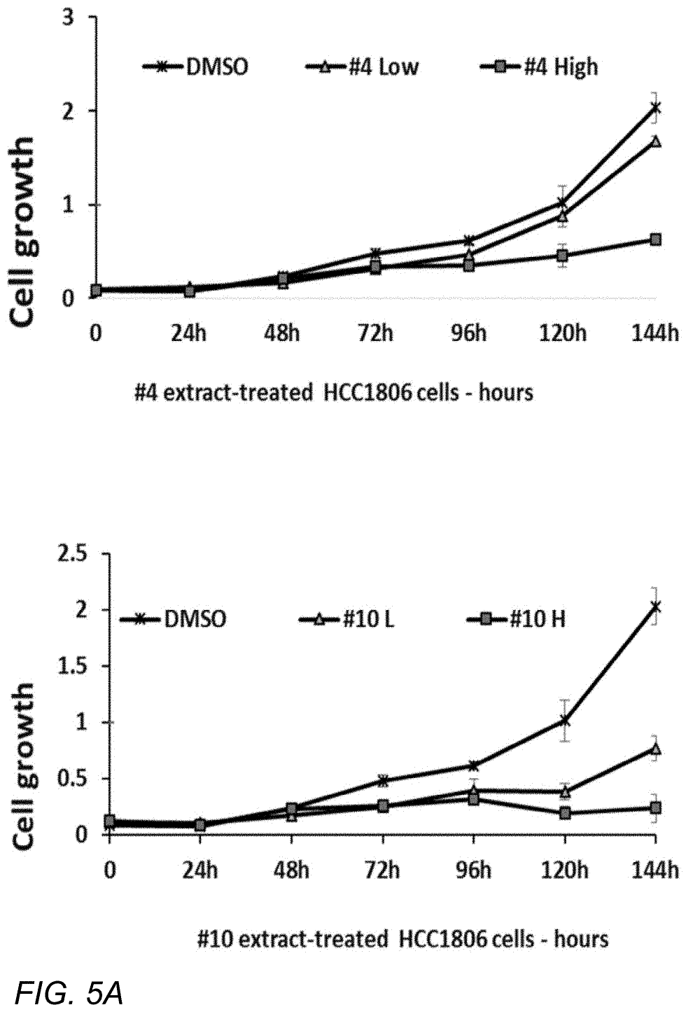

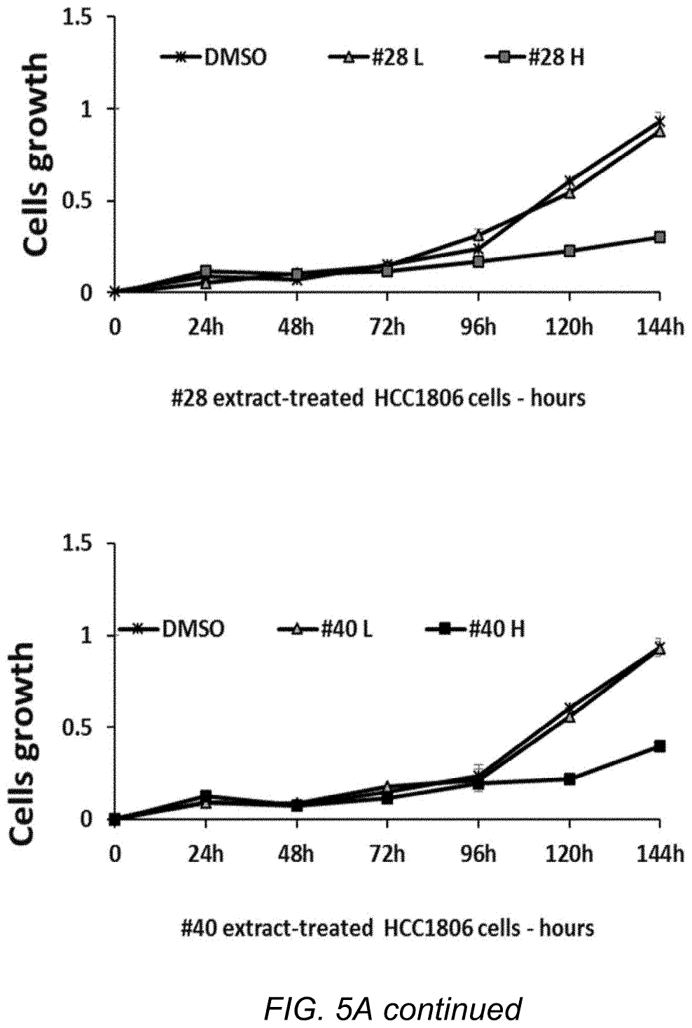

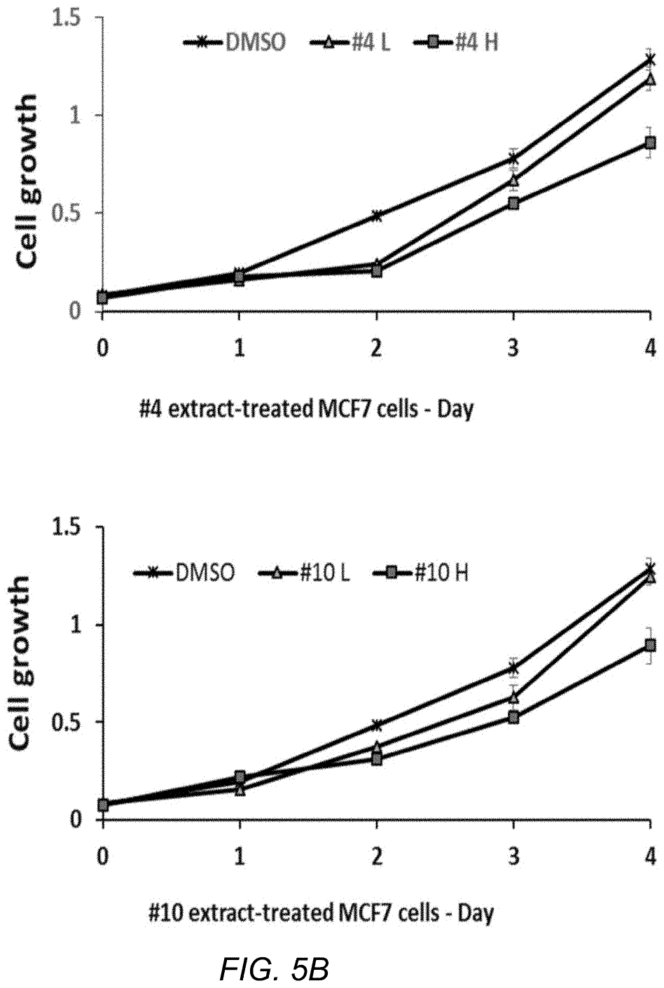

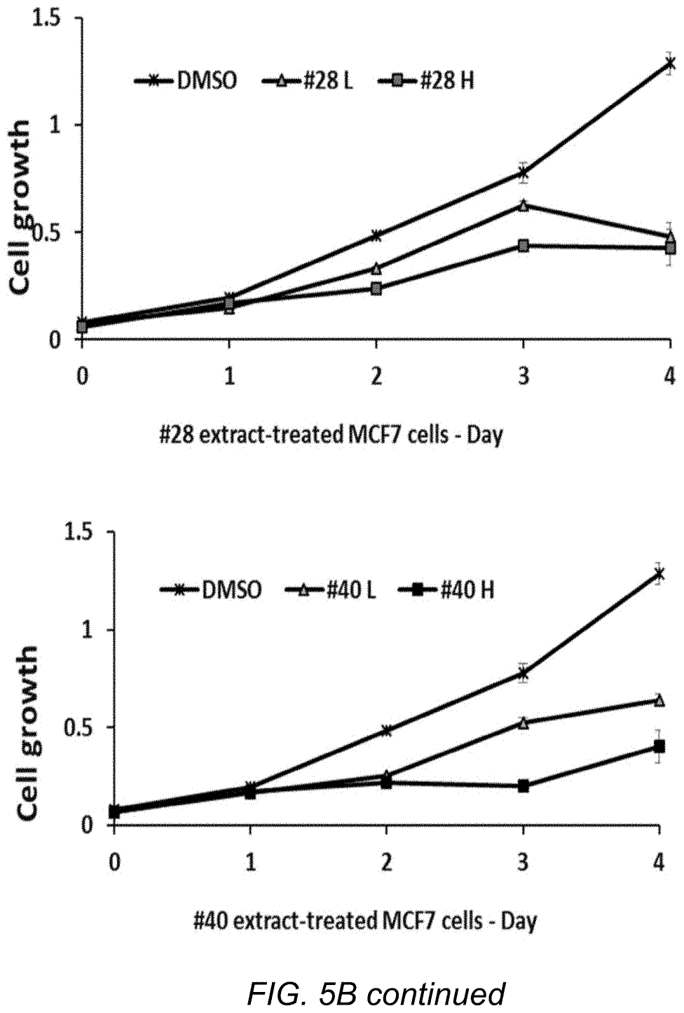

FIG. 5A provides graphical results of effects of four Cannabis extracts (#4, #10, #28, and #40) on HCC1806 breast cancer cell proliferation (arbitrary units of proliferation), in accordance with some embodiments of the present invention.

FIG. 5B provides graphical results of effects of four Cannabis extracts (#4, #10, #28, and #40) on MCF7 breast cancer cell proliferation (arbitrary units of proliferation), in accordance with some embodiments of the present invention.

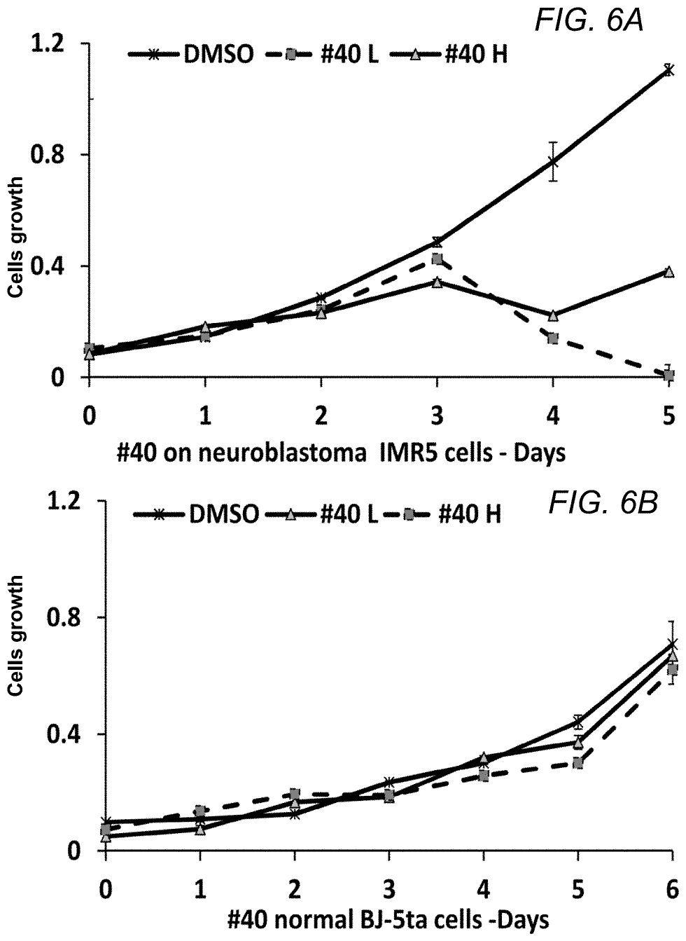

FIG. 6A provides graphical results of effects of line #40 Cannabis line extracts on suppression of neuroblastoma growth (arbitrary units of proliferation), but do not affect normal fibroblasts (FIG. 6B), in accordance with some embodiments of the present invention.

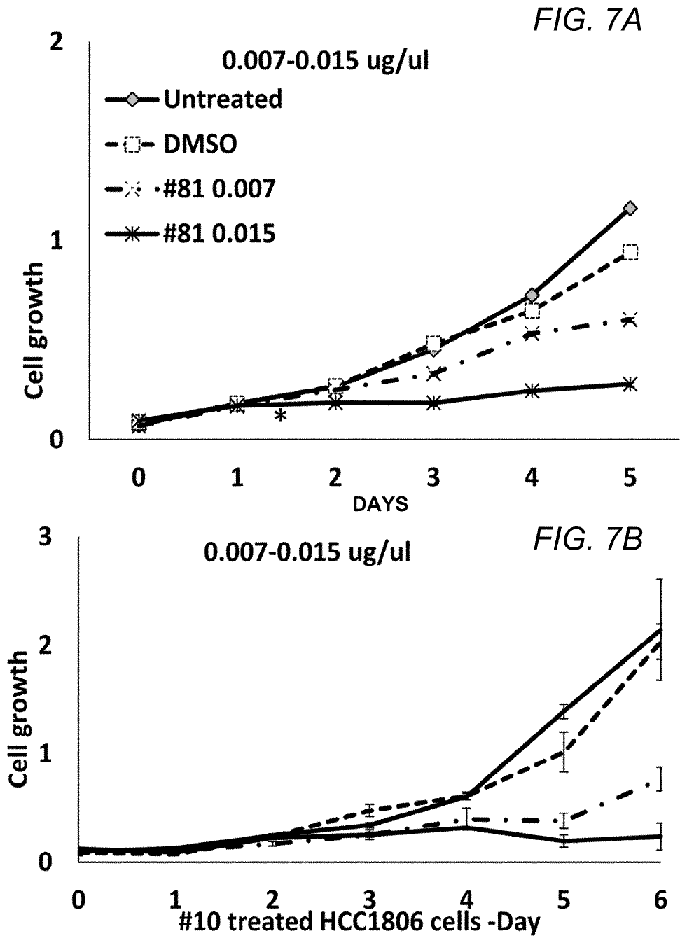

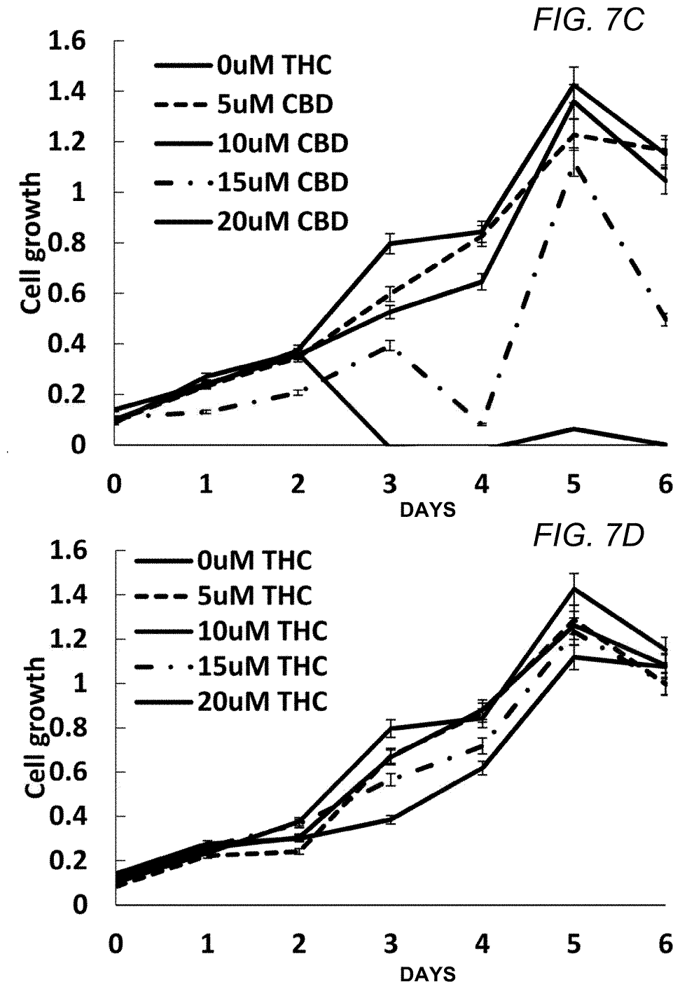

FIG. 7 provides graphical results of effects of extracts of Cannabis lines #81 (upper left) and #10 (lower left), containing ˜1% THC and ˜10% CBD, exhibit more potent anti-cancer effects (as show in arbitrary units of proliferation) as compared to pure cannabinoids THC and CBD, (right-hand panels) in accordance with some embodiments of the present invention.

The effect of pure CBD and pure THC were tested on HCC1806 cancer cells lines and identified active concentrations. CBD was effective at 15-20 μM, whereas THC was marginally effective at 20 μM. The effectiveness of pure cannabinoids and cannabinoids in the extracts were then tested.

At a concentration of THC of 10%, the amount of THC in the extracts with 0.007 μg/μl concentration is 0.0007 μg/μl, whereas in the extracts with 0.015 μg/μl concentration it is 0.0015 μg/μl. In comparison, pure THC at the amount of 0.0063 μg/μl (20 μM) was only marginally and insignificantly effective, making the extract of lower concentration of THC of 0.0007 μg/μl being over 9-fold more effective. Similarly, for CBD, at a concentration of CBD at 10%, there is 0.0007 μg/μl of CBD in 0.007 μg/μl extract and 0.0015 μg/μl in 0.015 μg/μl extract. Pure CBD was effective at concentration of 15 μM, which is 0.0047 μg/μl, making CBD in the extract being ˜7-fold more effective than pure CBD.

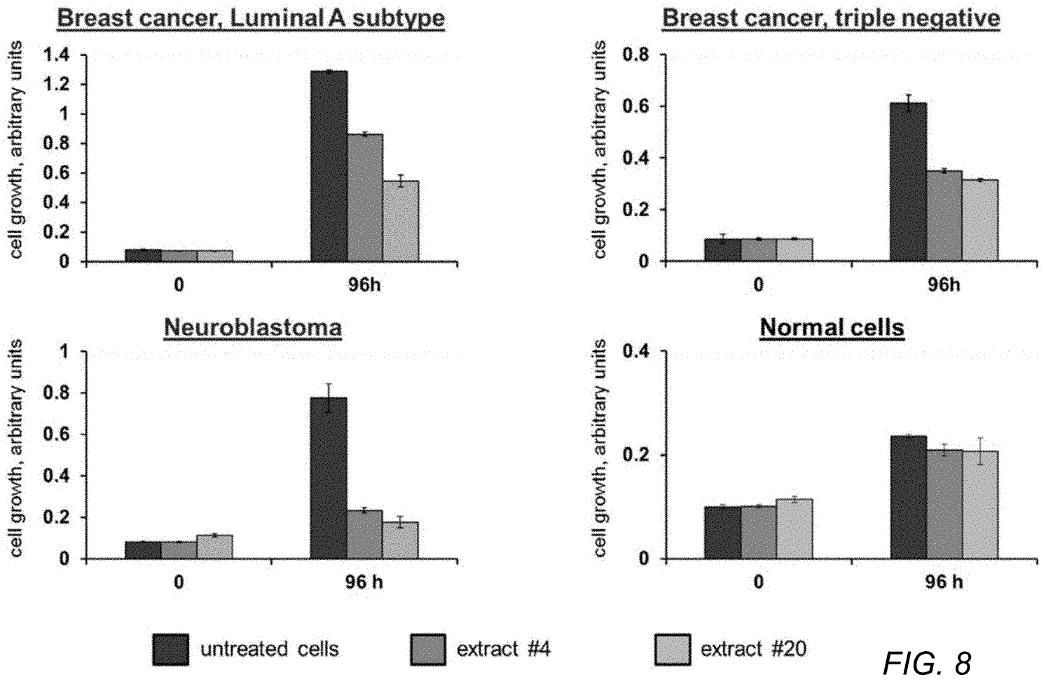

FIG. 8 shows graphical results of the effects of two different extracts Cannabis lines (#4 and #20) on the growth (arbitrary units of proliferation) of cancer cells (breast cancer, upper left panel, neuroblastoma lower left panel, and breast cancer triple negative, upper right panel), but not on normal cells (lower right panel), in accordance with some embodiments of the present invention.

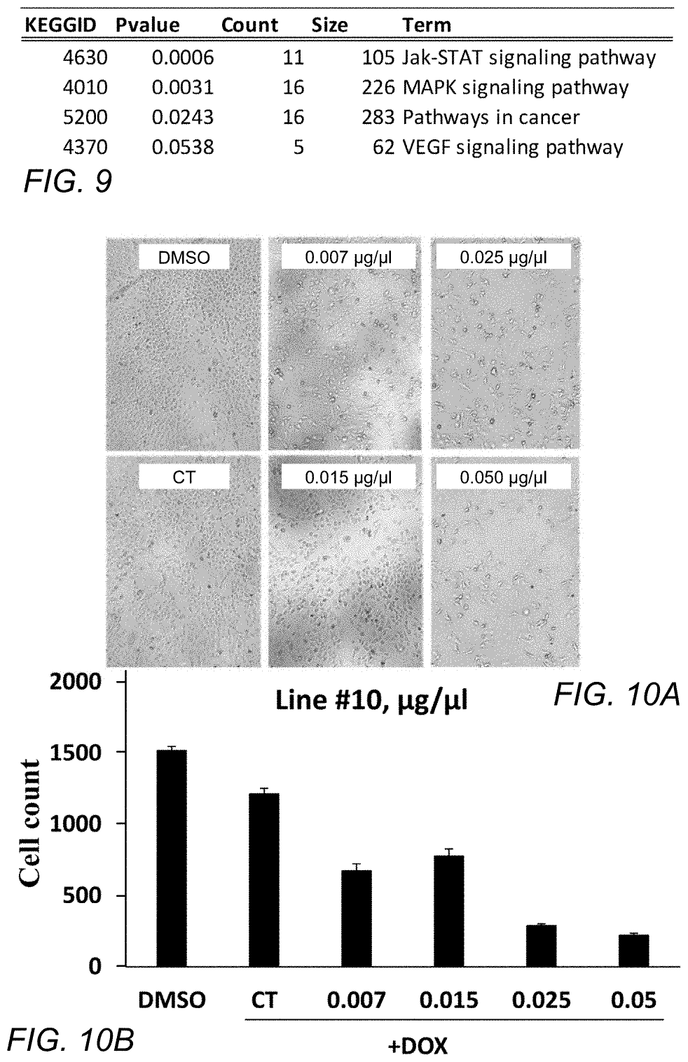

FIG. 9 shows an output table of modulation of gene expression in four different pathways in response to human skin tissues treatment with Cannabis line #4 after exposure to a known cancer causing agent (UV radiation), affecting pathways involved in cancer, apoptosis and metastasis. “KEGGID” shows the pathway ID (from KEGG pathway software), “P value” shows statistical significance, “Count” shows the number of genes altered in the pathway, “Size” shows total number of genes associated with a given pathway, “Term” shows the name of the pathway. A p value of less than 0.05 is considered to be significant, in accordance with some embodiments of the present invention.

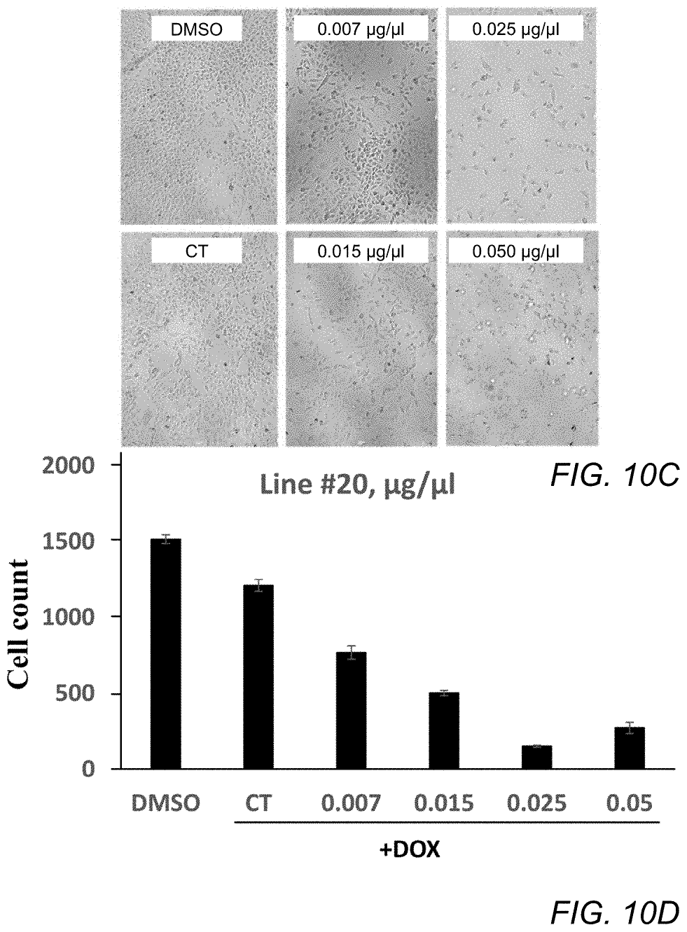

FIG. 10A shows images of MCF-7/DOX cells were treated with DOX (48 h, 1 μg/ml) in combination with Cannabis extract #10 at 0.007, 0.015, 0.025 and 0.05 μg/μl;

FIG. 10B—shows a cell count of MCF-7/DOX cells, which were treated with DOX (48 h, 1 μg/ml) in combination with Cannabis extract #10 at 0.007, 0.015, 0.025 and 0.05 μg/μl;

FIG. 10C—shows images of MCF-7/DOX cells, which were treated with DOX (48 h, 1 μg/ml) in combination with Cannabis extract #20 at 0.007, 0.015, 0.025 and 0.05 μg/μl;

FIG. 10D—cell count of MCF-7/DOX cells were treated with DOX (48 h, 1 μg/ml) in combination with Cannabis extract #20 at 0.007, 0.015, 0.025 and 0.05 μg/μl;

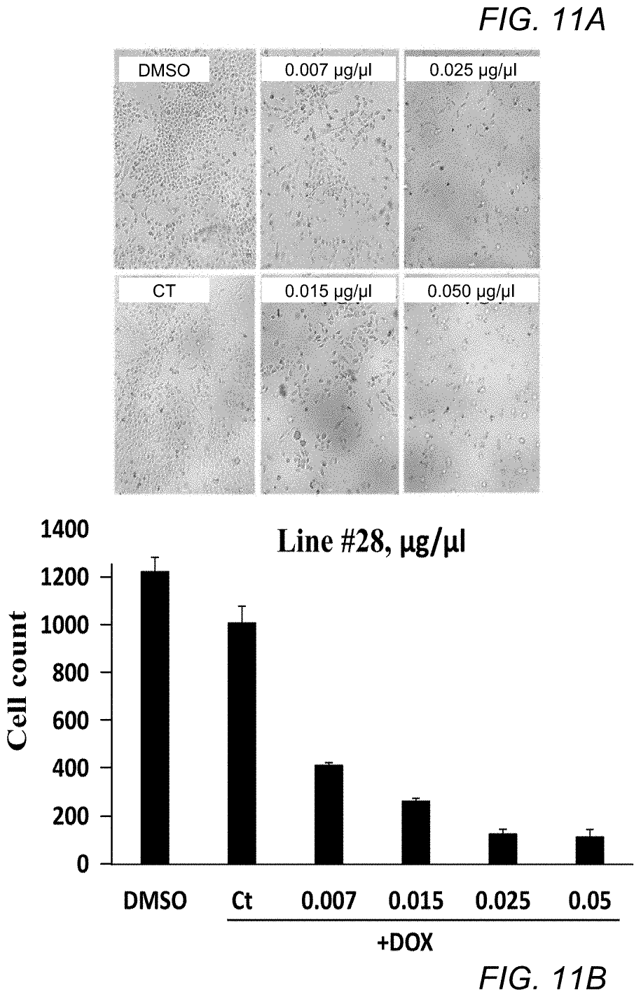

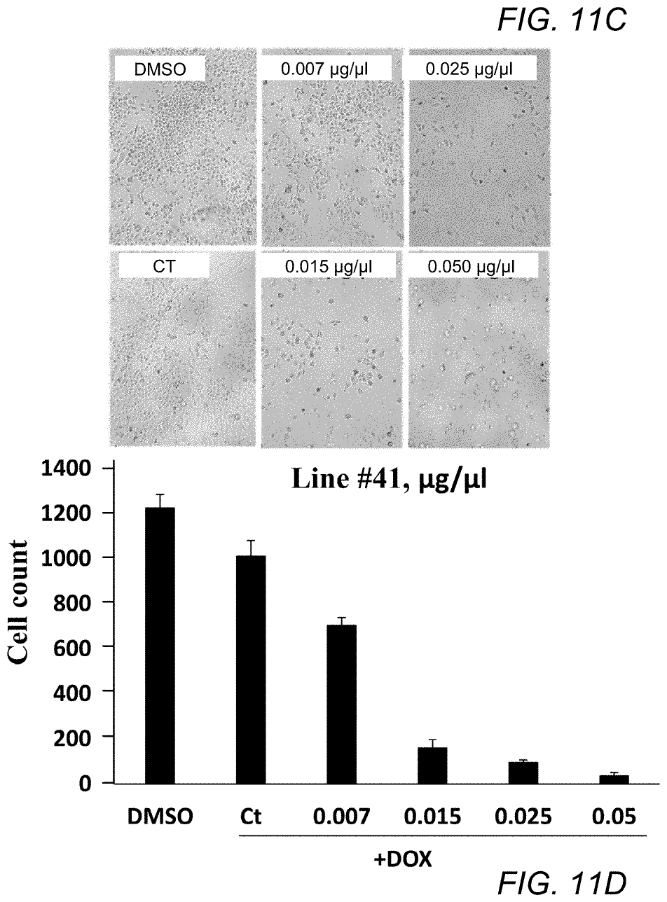

FIG. 11A—images of MCF-7/DOX cells were treated with DOX (48 h, 1 μg/ml) in combination with Cannabis extract #28 at 0.007, 0.015, 0.025 and 0.05 μg/μl,

FIG. 11B—cell count of MCF-7/DOX cells were treated with DOX (48 h, 1 μg/ml) in combination with Cannabis extract #28 at 0.007, 0.015, 0.025 and 0.05 μg/μl;

FIG. 11C—images of MCF-7/DOX cells were treated with DOX (48 h, 1 μg/ml) in combination with Cannabis extract #41 at 0.007, 0.015, 0.025 and 0.05 μg/μl;

FIG. 11D—cell count of MCF-7/DOX cells were treated with DOX (48 h, 1 μg/ml) in combination with Cannabis extract #41 at 0.007, 0.015, 0.025 and 0.05 μg/μl;

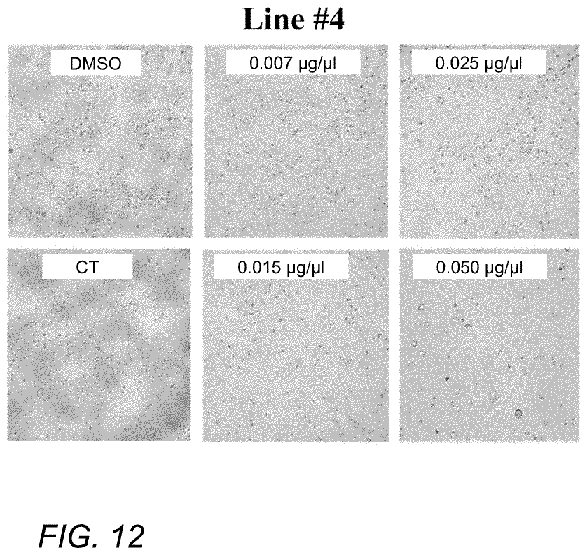

FIGS. 10A-10D, 11A-11D and 12 provide graphical results of the effect of Cannabis extracts #10 (FIGS. 10A-10B), #20 (FIGS. 10C-10D), #28 (FIGS. 11A-11B), #41 (FIGS. 11C-11D) and #4 (FIG. 12) on cell proliferation. Graphs were generated based on the photos taken from the live cells. Cells treated with 0.05 μg/μl of Cannabis extract #20 were harvested early at 24 hours due to observed cell death. The results are as follows, for Extract #28 (FIGS. 11A-11B), for extract #41 (FIGS. 11C-11D) and for extract #4 (FIG. 12), at concentrations of 0.007, 0.015, 0.025 and 0.05 μg/μl on doxorubicin resistant MCF-7/DOX cells. These figures show dose-dependent sensitivity of MCF-7/DOX cells co-treated for 48 h with 1 μg/ml doxorubicin. Upper panel (FIGS. 10A, 10C, 11A and 11C, and FIG. 12) shows the under the microscope picture of treated cells whereas the lower panel shows the quantification of images (FIGS. 10B and 10D, 11B and 11D).

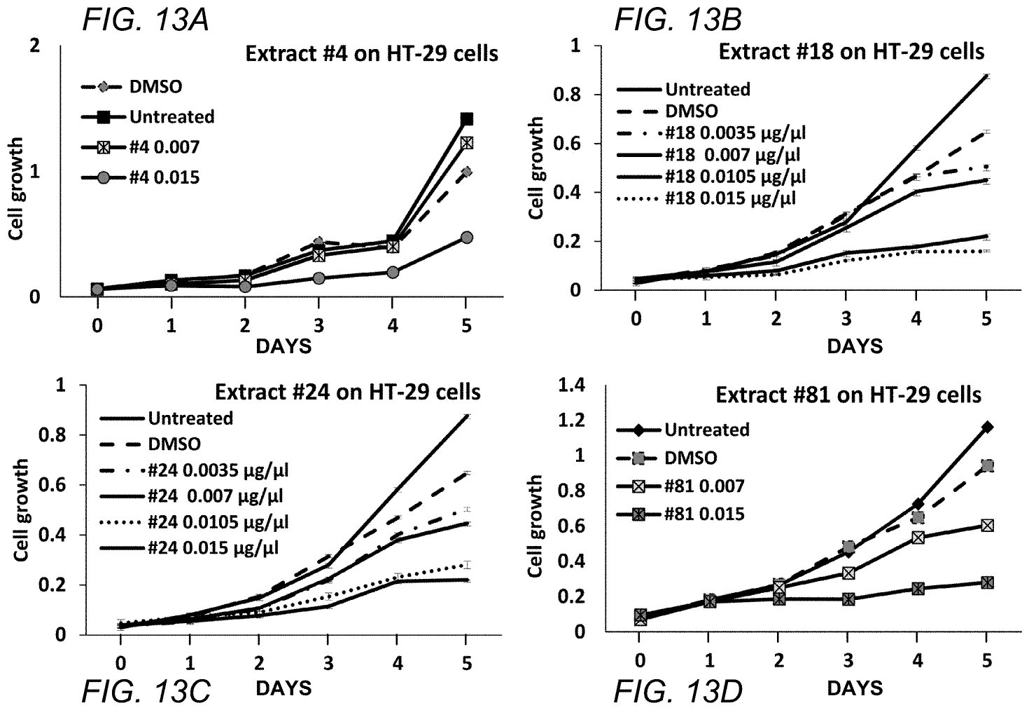

FIGS. 13A-13D show the dose dependent decrease in the proliferation (arbitrary units) of HT29 colorectal adenocarcinoma cells in response to #4, 18, #24 and #33 extracts.

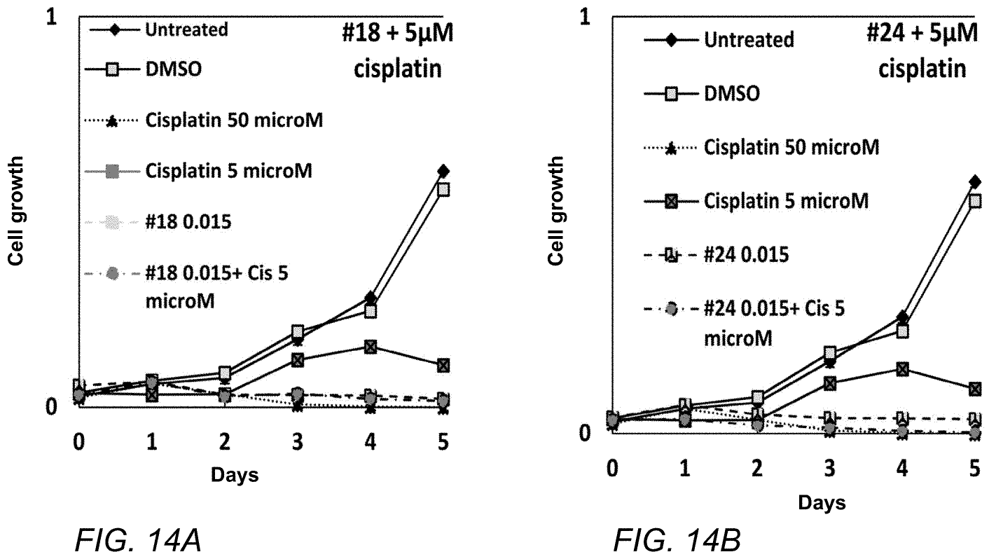

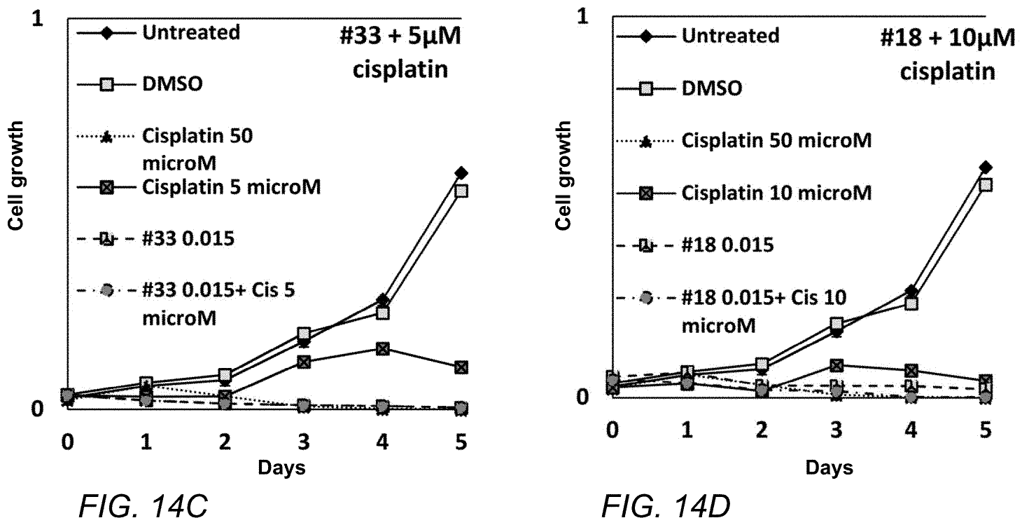

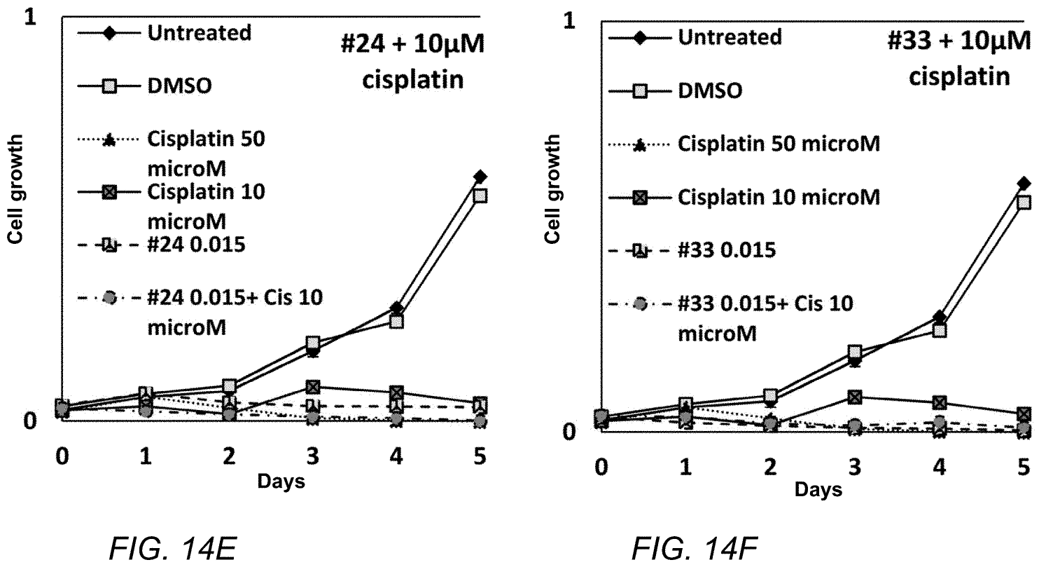

FIGS. 14A-14F show the potentiating effect in the decrease of proliferation (arbitrary units) of colon cancer cells HT29 when the cells are co-treated with 5 or 10 μM cisplatin and of the extracts—#18, #24 or #33 at a concentration of 0.015 μg/μl.

FIGS. 15A-15D show the decrease in the proliferation (arbitrary units) of neuroblastoma cells SK-N-MC treated with extracts #4, #10, #20 and #28 at a concentration of 0.007 (low) and 0.015 (high) μg/μl.

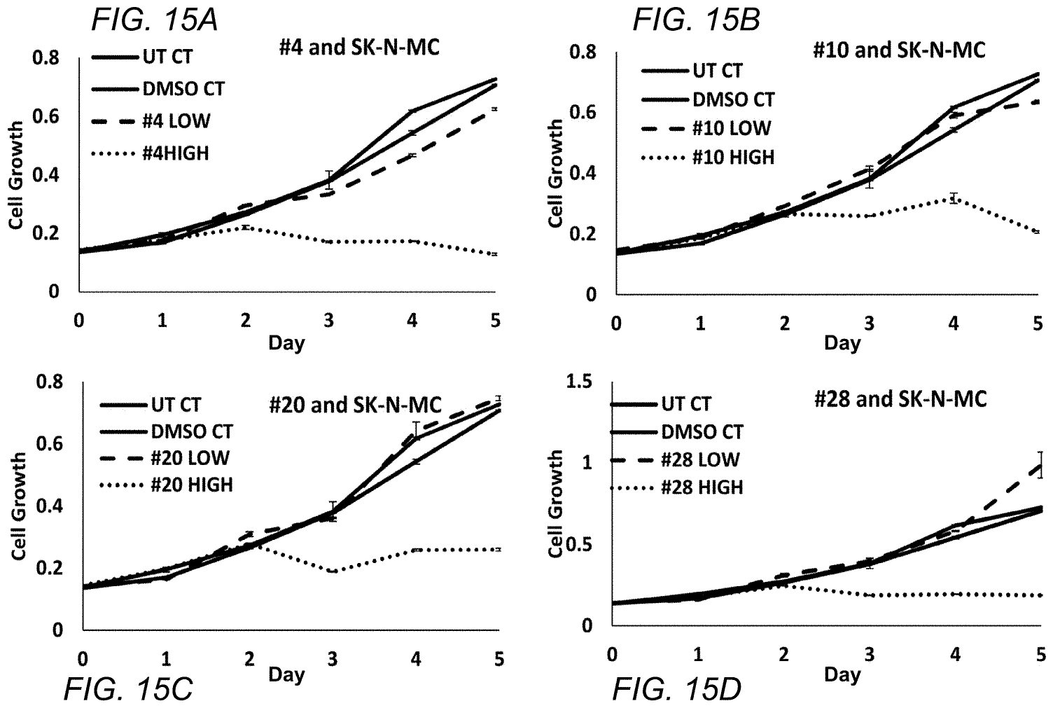

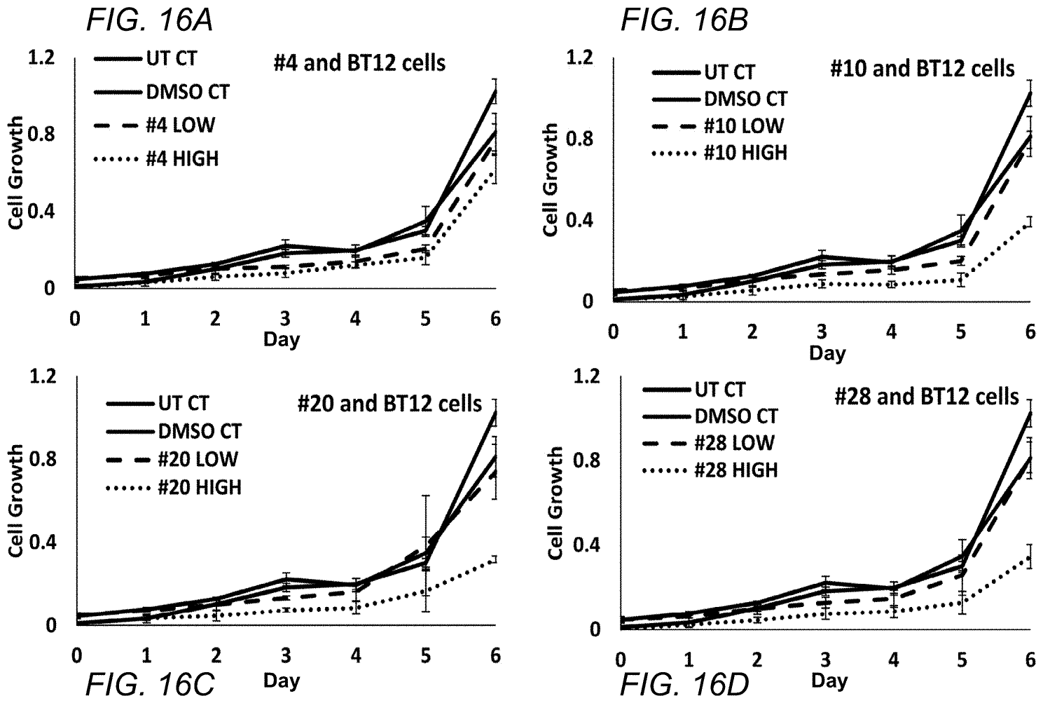

FIGS. 16A-16D show the decrease in the proliferation (arbitrary units) of atypical teratoid/rhabdoid tumor cells BT-12 treated with extracts #4, #10, #20 and #28 at a concentration of 0.007 (low) and 0.015 (high) μg/μl.

FIGS. 17A-17D show the decrease in the proliferation (arbitrary units) of atypical teratoid/rhabdoid tumor cells BT-16 treated with extracts #4, #10, #20 and #28 at a concentration of 0.007 (low) and 0.015 (high) μg/μl.

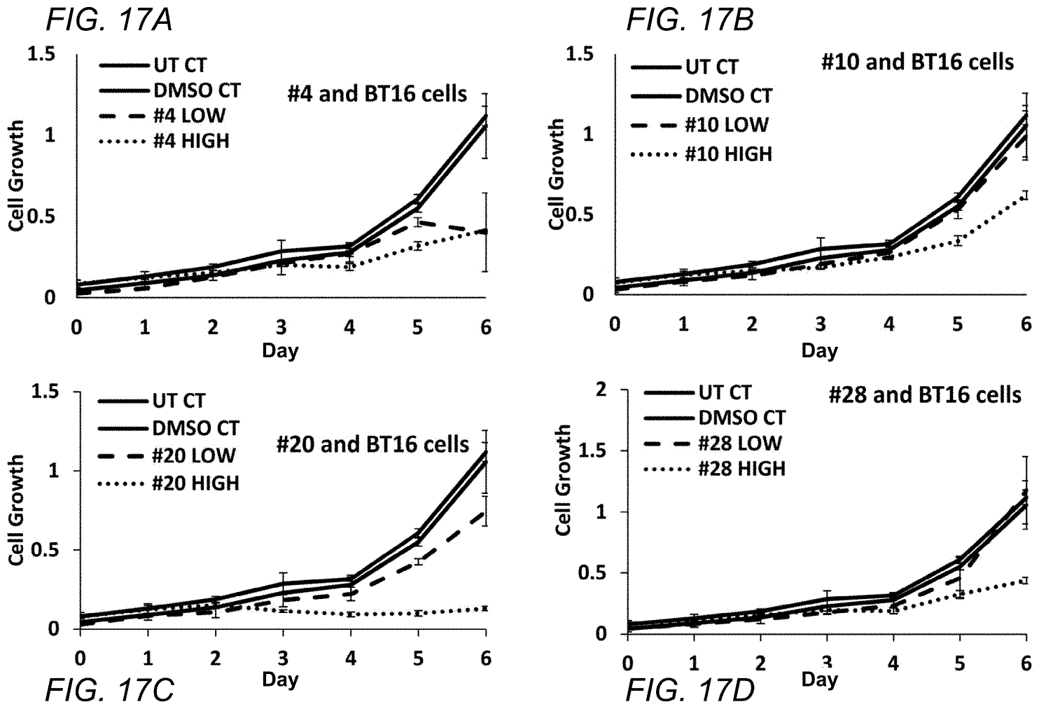

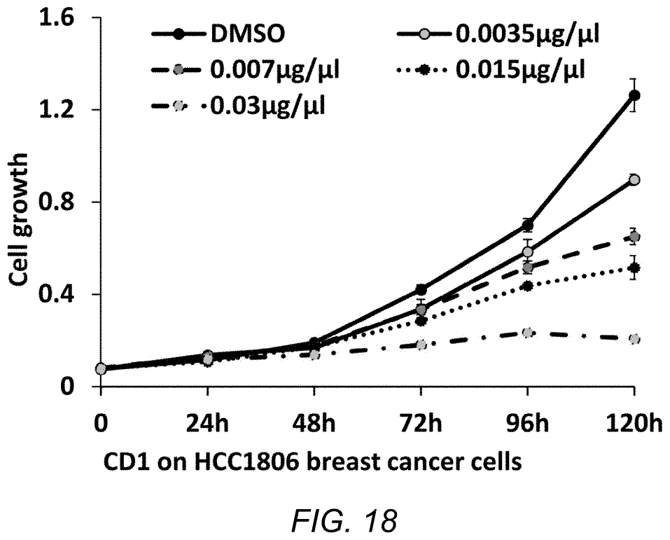

FIG. 18 shows the decrease in the proliferation (arbitrary units) of breast cancer cell HCC1806 treated with CD10 extract at four different concentrations.

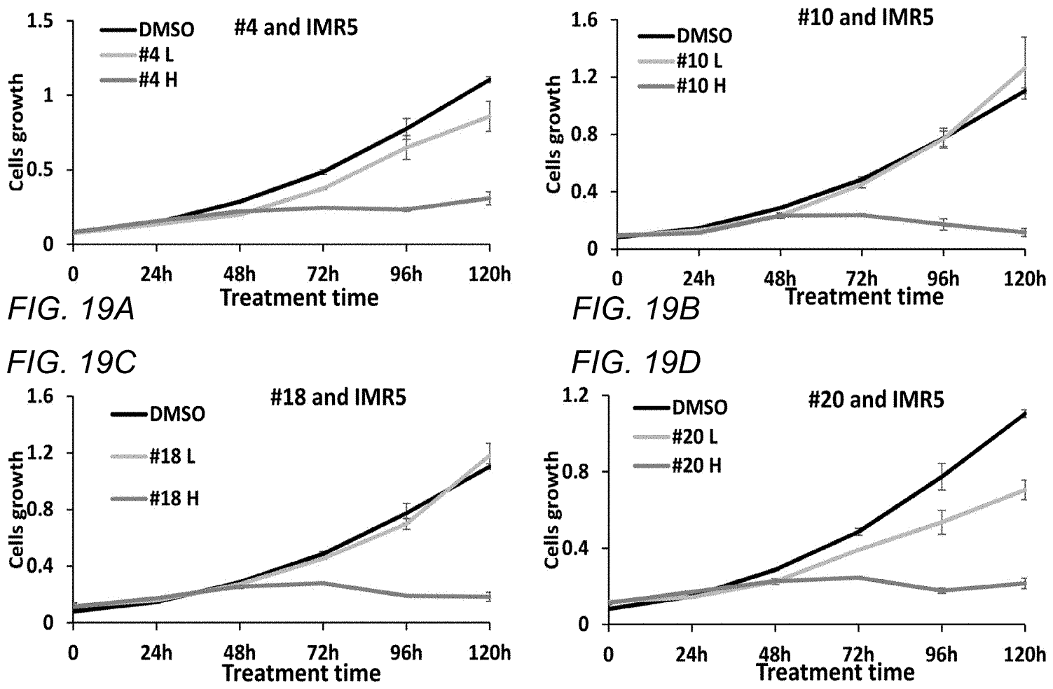

FIG. 19A-19D shows the decrease in the proliferation (arbitrary units) of neuroblastoma cells IMR5 cells treated with extracts #4, #10, #18, #20, #24, #30 and #40 at a concentration of 0.007 (low) and 0.015 (high) μg/μl.

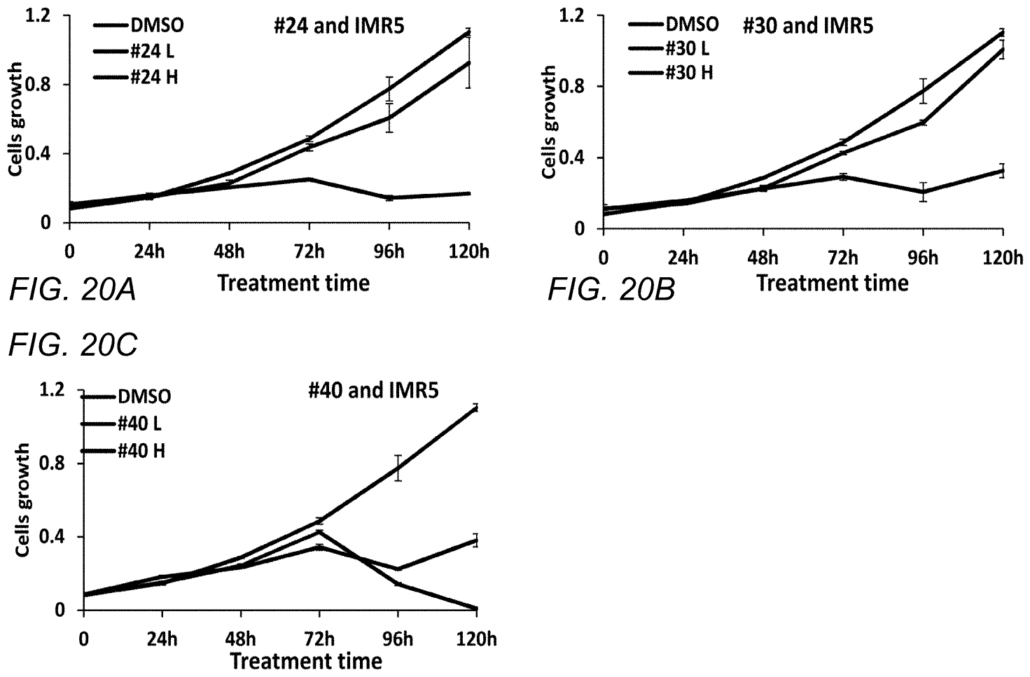

FIGS. 20A-20C shows the decrease in the proliferation (arbitrary units) of neuroblastoma IMR5 cells in response to treatments with extracts #24, #30 and #40 at a concentration of 0.007 (low) and 0.015 (high) μg/μl, in accordance with some embodiments of the present invention.

Non-Limiting Examples of Cannabis and/or Hemp Plant Line Extracts' Biologicial Activity
1. CD10 strongly suppresses breast cancer cell proliferation by causing G2 or S phase cell cycle arrest in a dose-dependent manner 0.007 μg/μl and 0.015 μg/μl has no effect on normal fibroblast growth.
2. #4 strongly suppresses breast cancer cell proliferation by causing G2 or S phase cell cycle arrest in a dose-dependent manner 0.007 μg/μl #4 has no effect on normal fibroblast growth, whereas 0.015 μg/μl #4 may promote normal fibroblast growth.
3. #10 strongly suppresses breast cancer cell proliferation by causing S phase cell cycle arrest in a dose-dependent manner 0.007 μg/μl #10 has no effect on normal fibroblast growth, whereas 0.015 μg/μl #10 may promote normal fibroblast growth.
4. #18 strongly suppresses breast cancer cell proliferation by causing G1 or S phase cell cycle arrest in a dose-dependent manner 0.007 μg/μl #18 has no effect on normal fibroblast growth, whereas 0.015 μg/μl #18 may promote normal fibroblast growth.
5. #20 strongly suppresses breast cancer cell proliferation by causing G1 cell cycle arrest in a dose-dependent manner Both 0.007 μg/μl and 0.015 μg/μl #20 has no significant effect on normal fibroblast growth.
6. #24 strongly suppresses breast cancer cell proliferation by causing G1 or S phase cell cycle arrest in a dose-dependent manner 0.007 μg/μl of extract #24 has no effect on normal fibroblast growth, whereas 0.015 μg/μl #24 may promote normal fibroblast growth.
7. #28 strongly suppresses breast cancer cell proliferation by causing G1 or S phase cell cycle arrest in a dose-dependent manner 0.007 μg/μl of extract #28 has no effect on normal fibroblast growth, whereas 0.015 μg/μl of extract #28 may promote normal fibroblast growth.
8. #30 strongly suppresses breast cancer cell proliferation by causing G1 cell cycle arrest in a dose-dependent manner 0.007 μg/μl of extract #30 has no effect on normal fibroblast growth, whereas 0.015 μg/μl of extract #30 may promote normal fibroblast growth.
9. #40 strongly suppresses breast cancer cell proliferation by causing G2 cell cycle arrest in a dose-dependent manner 0.007 μg/μl of extract #40 has no effect on normal fibroblast growth, whereas 0.015 μg/μl of extract #40 may promote normal fibroblast growth.
10. #4, #10, #20, and #28 strongly suppress neuroblastoma cell growth in a dose-dependent manner; high doses (0.015 μg/μl) were most effective.
11. #4, #10, #20, and #28 strongly suppress Ewing sarcoma cell growth in a dose-dependent manner; high doses (0.015 μg/μl) were most effective.
12. #4, #10, #20, and #28 strongly suppress ATRT cell growth in a dose-dependent manner; high doses (0.015 μg/μl) were most effective.
13. #4 profoundly affects gene expression in the normal skin tissues exposed to a well-established cancer-causing agent—UV. #4 affected expression of genes and pathways involved in cancer, cell growth, proliferation and metastasis in a manner that indicates protective, anti-cancer and anti-metastatic effects of the extract.
14. #4 strongly suppresses proliferation of chemo-resistant (resistant to doxorubicin) breast cancer cells. Concentrations from 0.007 to 0.05 μg/μl resulted in dose-dependent decrease in proliferation of doxorubicin-resistant cells.
15. #10 strongly suppresses proliferation of chemo-resistant (resistant to doxorubicin) breast cancer cells. Concentrations from 0.007 to 0.05 μg/μl resulted in dose-dependent decrease in proliferation of doxorubicin-resistant cells.
16. #20 strongly suppresses proliferation of chemo-resistant (resistant to doxorubicin) breast cancer cells. Concentrations from 0.007 to 0.05 μg/μl resulted in dose-dependent decrease in proliferation of doxorubicin-resistant cells.
17. #28 strongly suppresses proliferation of chemo-resistant (resistant to doxorubicin) breast cancer cells. Concentrations from 0.007 to 0.05 μg/μl resulted in dose-dependent decrease in proliferation of doxorubicin-resistant cells.
18. #41 strongly suppresses proliferation of chemo-resistant (resistant to doxorubicin) breast cancer cells. Concentrations from 0.007 to 0.05 μg/μl resulted in dose-dependent decrease in proliferation of doxorubicin-resistant cells.
19. #4 strongly suppresses proliferation of HT29 colon cancer cells.
20. #18 strongly suppresses proliferation of HT29 colon cancer cells.
21. #24 strongly suppresses proliferation of HT29 colon cancer cells.
22. #33 strongly suppresses proliferation of HT29 colon cancer cells.
23. #18 at a concentration of 0.015 μg/μl potentiates the effect of 5 and 10 μM cisplatin on HT29 colon cancer cells.
24. #24 at a concentration of 0.015 μg/μl potentiates the effect of 5 and 10 μM cisplatin on HT29 colon cancer cells.
25. #33 at a concentration of 0.015 μg/μl potentiates the effect of 5 and 10 μM cisplatin on HT29 colon cancer cells.
26. #4 inhibit proliferation of SK-N-MC neuroblastoma cells in dose dependent manner
27. #10 inhibit proliferation of SK-N-MC neuroblastoma cells in dose dependent manner
28. #20 inhibit proliferation of SK-N-MC neuroblastoma cells in dose dependent manner
29. #28 inhibit proliferation of SK-N-MC neuroblastoma cells in dose dependent manner
30. #4 at a concentration of 0.007 and 0.015 μg/μl inhibit the proliferation of atypical teratoid/rhabdoid tumor cells BT-12.
31. #10 at a concentration of 0.007 and 0.015 μg/μl inhibit the proliferation of atypical teratoid/rhabdoid tumor cells BT-12.
32. #20 at a concentration of 0.007 and 0.015 μg/μl inhibit the proliferation of atypical teratoid/rhabdoid tumor cells BT-12.
33. #28 at a concentration of 0.007 and 0.015 μg/μl inhibit the proliferation of atypical teratoid/rhabdoid tumor cells BT-12
34. #4 at a concentration of 0.007 and 0.015 μg/μl inhibit the proliferation of atypical teratoid/rhabdoid tumor cells BT-16.
35. #10 at a concentration of 0.007 and 0.015 μg/μl inhibit the proliferation of atypical teratoid/rhabdoid tumor cells BT-16
36. #20 at a concentration of 0.007 and 0.015 μg/μl inhibit the proliferation of atypical teratoid/rhabdoid tumor cells BT-16.
37. #28 at a concentration of 0.007 and 0.015 μg/μl inhibit the proliferation of atypical teratoid/rhabdoid tumor cells BT-16
38. #4 at a concentration of 0.007 and 0.015 μg/μl inhibit the proliferation of neuroblastoma cells IMR5.
39. #10 at a concentration of 0.007 and 0.015 μg/μl inhibit the proliferation of neuroblastoma cells IMR5.
40. #18 at a concentration of 0.007 and 0.015 μg/μl inhibit the proliferation of neuroblastoma cells IMR5.
41. #20 at a concentration of 0.007 and 0.015 μg/μl inhibit the proliferation of neuroblastoma cells IMR5.
42. #24 at a concentration of 0.007 and 0.015 μg/μl inhibit the proliferation of neuroblastoma cells IMR5.
43. #30 at a concentration of 0.007 and 0.015 μg/μl inhibit the proliferation of neuroblastoma cells IMR5.
44. #40 at a concentration of 0.007 and 0.015 μg/μl inhibit the proliferation of neuroblastoma cells IMR5.

Materials and Methods

Plant Crude Extract Preparation:

Solvent used: ethyl acetate ACS grade from Fisher cat # E145-4 (99.9% pure)

Extract Preparation: 3 g of the powdered plant tissue were weighed using an analytical balance. Plant material was placed inside a 250 mL Erlenmeyer flask (clean). 100 mL of Ethyl Acetate was poured into the flask containing the plant material. The flasks were then wrapped with tin foil and shaken continuously (120 rpm) in an incubator at 21° C. overnight and in the dark.

After overnight solvent extraction the extracts were filtered through cotton into a 100 mL round bottom flask. The extracts were concentrated to around 2-3 mL using a rotary vacuum evaporator. The extracts were then transferred to a tared 3 dram vial (cat #60975L Kimble obtained from Fisher Scientific).

The leftover solvent was evaporated to dryness in an oven overnight at 50° C. to eliminate the solvent completely. Mass of each extract was recorded.

Bioassay Preparation:

Preparation of 60 mg/mL Stock solutions.

The stocks were prepared weighing a 3-6 mg of crude extract into a micro centrifuge tube. The crude extract was dissolved in DMSO (Dimethyl sulfoxide anhydrous from Life technologies cat # D12345) to reach 60 mg/mL final concentration and stored at −20° C.

Preparation of Crude Extracts for Bioassay.

Appropriate cell culture media (in our experiments RPMI+10% FBS or EMEM+10% FBS) was used to dilute the 60 mg/mL stock. The stocks are allowed to thaw then added to the cell culture media, mixed thoroughly to ensure they are in solution and filtered through a 0.22 um syringe filter. These filtrates were ready to be applied to cells and tested.

Cannabis Extracts' Preparation

1) Add 2.45 μl of stock extract (60 mg/ml) to 21 ml of medium to make a working medium containing 0.007 mg/ml extract.

2) Add 5.25 μl of stock extract (60 mg/ml) to 21 ml of medium to make a working medium containing 0.015 mg/ml extract.

3) Sterilize using 0.22 um filter.

All Cannabis extracts were dissolved in DMSO.

MTT Assay

Cell Lines Used

MCF7 (ATCC® HTB-22™)—mammary gland, breast; derived from metastatic site: pleural effusion

MCF7/DOX-MCF7 cells resistant to Doxorubicin

Both MCF7 and MCF7/DOX use Eagle's Minimum Essential Medium (EMEM) (ATCC 30-2003)

+10% FBS (Fetal Bovine Serum, Sigma Aldrich 12103C-500ML)

+0.01 mg/ml human recombinant insulin (19278 Sigma-Aldrich Insulin solution human, 5ML)

BJ-5ta (ATCC® CRL4001™) Fibroblast immortalized with hTERT

Complete medium-Dulbecco's Modified Eagle's Medium (DMEM) from ATCC+10% FBS (Fetal Bovine Serum, Sigma Aldrich 12103C-500ML)

HMEC (ATCC® PCS600010™)—Primary Mammary Epithelial Cells; Normal, Human

Complete medium-Mammary Epithelial Cell Basal Medium (ATCC® PCS-600030™)+Mammary Epithelial Cell Growth Kit (ATCC® PCS-600-040™)+1% penicillin/streptomycin (Thermo Fisher Catalog number: 15140122)

IMR5 neuroblastoma cells

Complete Medium-RPMI 1640 (Roswell Park Memorial Institute medium ThermoFisher Catalog number: 11875093)+10% FBS (Fetal Bovine Serum, Sigma Aldrich 12103C-500ML)+1% penicillin/streptomycin (Thermo Fisher Catalog number: 15140122)+1% NEAA (MEM Non-Essential Amino Acids, gibco 11140-050 100 ml)

HT-29 (ATCC®HTB-38™)—Colon Colorectal Adenocarcinoma

Complete Medium-RPMI 1640 (Roswell Park Memorial Institute medium ThermoFisher Catalog number: 11875093)+10% FBS (Fetal Bovine Serum, Sigma Aldrich 12103C-500ML)+1% penicillin/streptomycin (Thermo Fisher Catalog number: 15140122)+1% NEAA (MEM Non-Essential Amino Acids, gibco 11140-050 100 ml)

HCC 1806—Primary Acantholytic Squamous Cell Carcinoma

Complete Medium—RPMI 1640 (e.g. Roswell Park Memorial Institute medium ThermoFisher Catalog number: 11875093)

+10% FBS (e.g. Fetal Bovine Serum, SigmaAldrich 12103C-500ML)+1% penicillin/streptomycin (e.g. ThermoFisher Catalog number: 15140122)

WI-38—Normal Cells

Complete Medium—EMEM (e.g. SigmaAldrich M2279 Eagle's minimal essential medium)+10% FBS.

Medium Preparation

Switch on the cell culture hood and—spray work surface with 70% ethanol. Prepare all equipment (Pipette tips, Pasteur pipettes, falcon tubes, cell culture plate etc.). Switch on the UV light for a 15 min. Then thaw 50 ml FBS using water bath and pre-warm medium using water bath 37° C. for a max 10 min Thereafter, add 50 ml of FBS to a 500 ml bottle of medium (final concentration: 10% FBS) and sterilize medium using 0.22 um filter.

Note: all items should be sprayed with 70% ethanol before transferring them under the sterile cell culture hood!

Cell Culture

Take 2 cryo-vials containing the frozen cells from −80° C. storage and immediately place in a 37° C. water bath. Quickly thaw the cells (<1 minute) by gently swirling the vial in the 37° C. water bath until there is just a small bit of ice left in the vial. Then transfer the vials in a cell culture hood. Before opening, spray the outside of the vials with 70% ethanol.

Add 8 ml pre-warmed complete growth medium to 15 ml falcon tube, then add thawed cells. Spin with centrifuge at 2000 rpm for 5 min at room temperature, make sure the centrifuge is balanced before operation. Spray tube with 70% ethanol and transfer the tube to the cell culture hood.

Aseptically decant the supernatant without disturbing the cell pellet and add 10 ml of medium to each cell culture plate. Thereafter, take 1 ml of medium from each cell culture plate, re-suspend the cell pellet in 15 ml tube and transfer lml of re-suspended cells to each cell culture plates, and mix nicely by moving plate in right-left and reverse-forward directions.

Place cell culture plates in a 37° C. incubator with 5% CO₂.

Checking Cells

Cells were checked microscopically daily to ensure they are healthy and growing as expected. Attached-growth cells should attach to the bottom of the plate, round and plump or elongated in shape and refracting light around their membrane. Medium should be clear and pinky-orange in color. The medium should be changed every 2 days. Then when cells' confluency is 50%, the medium should be changed every day.

Set Up MTT Assay

(Colorimetric assays using the tetrazolium salt thiazolyl blue, also termed MTT, after methyl-thiazolyl-tetrazolium)

Around 1500 cells are in placed in each well in 96 well plates, 10 extracts (tested 2 concentrations each, 0.007 mg/ml and 0.015 μg/μl) were added to separate wells. Untreated cells and DMSO-treated cells were used as controls in separate wells. Experiments were performed in triplicate. A total number of 9×10⁴ cells are required at each concentration. The well plates were purchased from Sigma Aldrich.

Riss T L, Moravec R A, Niles A L, et al. Cell Viability Assays. 2013 May 1 [Updated 2016 Jul. 1]. In: Sittampalam G S, Coussens N P, Brimacombe K, et al., editors. Assay Guidance Manual [Internet]. Bethesda (Md.): Eli Lilly & Company and the National Center for Advancing Translational Sciences; 2004-. Available from: https://www.ncbi.nlm nih.gov/books/NBK144065/When cells grew to a confluency of 80%, the cells were harvested using TRYPSIN −1 ml in each plate. Cells were transferred to a 15 ml tube with complete medium (total volume 10 ml) and mixed nicely. take 200 μl and add in 1.5 ml tube.

Cell number was quantified using a standard automated cell counter.
For example WI-38 cells—6.24×10⁵
6.24×10⁵=62.4×10⁴
9: 62.4=0.14 ml of cells
0.14 ml×11(10 extracts+1 control)=1.54 ml of total volume harvested cells
9−0.14=8.86 ml of medium
8.86 ml×11=97.46 ml of medium

Mix cells and medium nicely, add 150 μl per well, plate eight 96-well plates for each experiment.

Place 96-well plates in a 37° C. incubator with 5% CO₂. After 24 hours, the medium was changed to the medium containing extracts.

The MTT assay was performed from Day 0 to Day 7 by adding 15 μl MTT labeling reagent into each well and incubated at 37° C. for 4 hours. Thereafter, 150 μl solubilization solution was added into each well and incubated overnight in the 37° C. incubator with 5% CO₂.

3-(4,5-Dimethylthiazol-2-yl)-2,5-diphenyltetrazolium bromide assays were performed using the Cell Proliferation Kit I (Roche Diagnostics GmbH) as the manufacturer's instructions. The absorbance of the samples was measured at 595 nm using a microtiter plate reader (FLUOstar Omega, BMG LABTECH). The experiments were done in triplicate.

Cell-Cycle Assay on HCC1806 Cell Line with Plant Extracts Protocol:

1. Human breast cancer cell line HCC1806 (ATCC® CRL-2335TM) was cultured in RPMI-1640 medium supplemented with 10% fetal bovine serum and 1% Penicillin-Streptomycin. Cells were maintained at 37° C. in 5% CO₂, and medium was changed every 1-3 days. When the cells reached 80% confluency, they were passaged to the six-well plate.

2. Cells were incubated at six-well plates, 37° C. in 5% CO₂ until the cells reached 70%-80% confluency, vehicle (DMSO) or the plant extracts at low (0.0007 μg/μl) and high (0.0015 μg/μl) concentrations were added to the medium for 48-hour treatment.

3. Cells were collected after 48-hour treated and washed twice by ice-cold Phosphate Buffered Saline (PBS).

4. Cell number was counted by Luna-IITM Automated Cell Counter with 0.4% Trypan Blue Stain. Cells were re-suspended at 2×10⁶ cells in lml ice-cold PBS.

5. Cell suspension was slowly, drop-by-drop added into 9 ml of ice-cold absolute ethanol in the polypropylene, stored at −20° C. for at least 24 hours before staining.

6. Cells were centrifuged at 200×g, 10 min, 4° C. to remove the ethanol and washed twice by ice-cold PBS.

7. The pellet was re-suspended in 500 μl PI/Triton X-100 staining solution.

(PI/Triton X-100 staining solution recipe: 10 ml of 0.1% (v/v) Triton X-100 in PBS add 2 mg

8. Cells were incubated at room temperature for 30 min.

9. Cell cycle analyses were performed using a BD FACSCanto II Flow Cytometer (BD Biosciences) with propidium iodide staining solution (BD Biosciences) according to the manufacturer's instructions. All the experiments were done in duplicate.

The present invention further provides new unique Cannabis lines, extracts, dried powders from the extracts, compositions comprising the powders or parts thereof, compounds derived therefrom, pharmaceutical compositions comprising the compound(s) and methods for their use in anti-cancer therapies and modalities. The method includes generation of unique lines, whole plant extract preparation, treating cancer cells and normal cells with extracts in amount sufficient to kill cancer cells while sparing normal (non-proliferative) ones. The modulation of cell proliferation, growth and death results in efficient elimination of cancer cells in response to the anti-cancer therapies and modalities of the present invention.

Dosage Forms

The compositions of the present invention may be provided in any suitable dosage form. According to some embodiments, the dosage form is an oral dosage form. Oral dosage forms comprise liquids (solutions, suspensions, and emulsions), semi-solids (pastes), and solids (tablets, capsules, powders, granules, premixes, and medicated blocks).

Some examples of oral dosage forms in the art include, WO90/04391, which discloses an oral dosage form of omega-3 polyunsaturated acids to overcome the problems of diseases. It is known to supply said acids in soft gelatine capsule shells. EP 2 240 581 B1 discloses a gelatine capsule for pharmaceutical use with a controlled release of active ingredients and a process for the preparation of said gelatine capsules. During said process xylose is added to the liquid gelatine from which afterwards gelatine capsules are formed. Gelatine capsules manufactured according to the process provide retarded release of active ingredients.

U.S. Pat. No. 7,264,824 discloses and oral dosage form for food and food supplements, as well as dietetics comprising polyunsaturated acids in a xylose-hardened gelatine capsule with a retarded release time.

According to some embodiments of the present invention, the compositions described herein may be in a suspension or emulsion.

A suspension is a coarse dispersion of insoluble drug particles, generally with a diameter exceeding 1 um, in a liquid (usually aqueous) medium. Suspensions are useful for administering insoluble or poorly soluble drugs/components or in situations when the presence of a finely divided form of the material in the GI tract is required. The taste of most drugs is less noticeable in suspension than in solution, due to the drug being less soluble in suspension. Particle size is an important determinant of the dissolution rate and bioavailability of drugs in suspension. In addition to the excipients described above for solutions, suspensions include surfactants and thickening agents. Surfactants wet the solid particles, thereby ensuring the particles disperse readily throughout the liquid. Thickening agents reduce the rate at which particles settle to the bottom of the container. Some settling is acceptable, provided the sediment can be readily dispersed when the container is shaken. Because hard masses of sediment do not satisfy this criterion, caking of suspensions is not acceptable.

An emulsion is a system consisting of 2 immiscible liquid phases, one of which is dispersed throughout the other in the form of fine droplets; droplet diameter generally ranges from 0.1-100 um. The 2 phases of an emulsion are known as the dispersed phase and the continuous phase. Emulsions are inherently unstable and are stabilized through the use of an emulsifying agent, which prevents coalescence of the dispersed droplets. Creaming, as occurs with milk, also occurs with pharmaceutical emulsions. However, it is not a serious problem because a uniform dispersion returns upon shaking. Creaming is, nonetheless, undesirable because it is associated with an increased likelihood of the droplets coalescing and the emulsion breaking. Other additives include buffers, antioxidants, and preservatives. Emulsions for oral administration are usually oil (the active ingredient) in water, and facilitate the administration of oily substances such as castor oil or liquid paraffin in a more palatable form.

A paste is a 2-component semi-solid in which drug is dispersed as a powder in an aqueous or fatty base. The particle size of the active ingredient in pastes can be as large as 100 um. The vehicle containing the drug may be water; a polyhydroxy liquid such as glycerin, propylene glycol, or polyethylene glycol; a vegetable oil; or a mineral oil. Other formulation excipients include thickening agents, cosolvents, adsorbents, humectants, and preservatives. The thickening agent may be a naturally occurring material such as acacia or tragacanth, or a synthetic or chemically modified derivative such as xanthum gum or hydroxypropylmethyl cellulose. The degree of cohesiveness, plasticity, and syringeability of pastes is attributed to the thickening agent. It may be necessary to include a cosolvent to increase the solubility of the drug. Syneresis of pastes is a form of instability in which the solid and liquid components of the formulation separate over time; it is prevented by including an adsorbent such as microcrystalline cellulose. A humectant (eg, glycerin or propylene glycol) is used to prevent the paste that collects at the nozzle of the dispenser from forming a hard crust. Microbial growth in the formulation is inhibited using a preservative. It is critical that pastes have a pleasant taste or are tasteless.

A tablet consists of one or more active ingredients and numerous excipients and may be a conventional tablet that is swallowed whole, a chewable tablet, or a modified-release tablet (more commonly referred to as a modified-release bolus due to its large unit size). Conventional and chewable tablets are used to administer drugs to dogs and cats, whereas modified-release boluses are administered to cattle, sheep, and goats. The physical and chemical stability of tablets is generally better than that of liquid dosage forms. The main disadvantages of tablets are the bioavailability of poorly water-soluble drugs or poorly absorbed drugs, and the local irritation of the GI mucosa that some drugs may cause.

A capsule is an oral dosage form usually made from gelatin and filled with an active ingredient and excipients. Two common capsule types are available: hard gelatin capsules for solid-fill formulations, and soft gelatin capsules for liquid-fill or semi-solid-fill formulations. Soft gelatin capsules are suitable for formulating poorly water-soluble drugs because they afford good drug release and absorption by the GI tract. Gelatin capsules are frequently more expensive than tablets but have some advantages. For example, particle size is rarely altered during capsule manufacture, and capsules mask the taste and odor of the active ingredient and protect photolabile ingredients.

A powder is a formulation in which a drug powder is mixed with other powdered excipients to produce a final product for oral administration. Powders have better chemical stability than liquids and dissolve faster than tablets or capsules because disintegration is not an issue. This translates into faster absorption for those drugs characterized by dissolution rate-limited absorption. Unpleasant tastes can be more pronounced with powders than with other dosage forms and can be a particular concern with in-feed powders, in which it contributes to variable ingestion of the dose. Moreover, sick animals often eat less and are therefore not amenable to treatment with in-feed powder formulations. Drug powders are principally used prophylactically in feed, or formulated as a soluble powder for addition to drinking water or milk replacer. Powders have also been formulated with emulsifying agents to facilitate their administration as liquid drenches.

A granule is a dosage form consisting of powder particles that have been aggregated to form a larger mass, usually 2-4 mm in diameter. Granulation overcomes segregation of the different particle sizes during storage and/or dose administration, the latter being a potential source of inaccurate dosing. Granules and powders generally behave similarly; however, granules must deaggregate prior to dissolution and absorption.

A premix is a solid dosage form in which an active ingredient, such as a coccidiostat, production enhancer, or nutritional supplement, is formulated with excipients. Premix products are mixed homogeneously with feed at rates (when expressed on an active ingredient basis) that range from a few milligrams to ˜200 g/ton of food/beverage The density, particle size, and geometry of the premix particles should match as closely as possible those of the feed in which the premix will be incorporated to facilitate uniform mixing. Issues such as instability, electrostatic charge, and hygroscopicity must also be addressed. The excipients present in premix formulations include carriers, liquid binders, diluents, anti-caking agents, and anti-dust agents. Carriers, such as wheat middlings, soybean mill run, and rice hulls, bind active ingredients to their surfaces and are important in attaining uniform mixing of the active ingredient. A liquid binding agent, such as a vegetable oil, should be included in the formulation whenever a carrier is used. Diluents increase the bulk of premix formulations, but unlike carriers, do not bind the active ingredients. Examples of diluents include ground limestone, dicalcium phosphate, dextrose, and kaolin. Caking in a premix formulation may be caused by hygroscopic ingredients and is addressed by adding small amounts of anti-caking agents such as calcium silicate, silicon dioxide, and hydrophobic starch. The dust associated with powdered premix formulations can have serious implications for both operator safety and economic losses, and is reduced by including a vegetable oil or light mineral oil in the formulation. An alternate approach to overcoming dust is to granulate the premix formulation.

A medicated block is a compressed feed material that contains an active ingredient, such as a drug, anthelmintic, surfactant (for bloat prevention), or a nutritional supplement, and is commonly packaged in a cardboard box. Ruminants typically have free access to the medicated block over several days, and variable consumption may be problematic. This concern is addressed by ensuring the active ingredient is nontoxic, stable, palatable, and preferably of low solubility. In addition, excipients in the formulation modulate consumption by altering the palatability and/or the hardness of the medicated block. For example, molasses increases palatability and sodium chloride decreases it. Additionally, the incorporation of a binder such as lignin sulfonate in blocks manufactured by compression or magnesium oxide in blocks manufactured by chemical reaction, increases hardness. The hygroscopic nature of molasses in a formulation may also impact the hardness of medicated blocks and is addressed by using appropriate packaging.

In another embodiment, the composition of the present invention is in a chewable oral dosage form. In another embodiment, the chewable oral dosage form is a chewable tablet. In another embodiment, the chewable tablet of the invention is taken slowly by chewing or sucking in the mouth. In another embodiment, the chewable tablet of the invention enables the dried Cannabis extracts contained therein to be orally administered without drinking.

According to some embodiments of the present invention, the composition may comprise any suitable flavor or combination of flavors.

The composition may further comprise other additives, coloring, emulsifiers. The flavors and additives may be of a natural, semi-synthetic, synthetic source or combinations thereof.

In another embodiment of the present invention, the composition further comprises fructose, sorbitol, microcrystalline cellulose, magnesium stearate, or any combination thereof. In another embodiment, the composition further comprises chamomile. In another embodiment, the composition further comprises ginger. In another embodiment, the composition further comprises peppermint. In another embodiment, the composition further comprises anise. In another embodiment, the composition further comprises fennel. In another embodiment, the composition further comprises thyme. In another embodiment, the composition further comprises Arsenicum album. In another embodiment, the composition further comprises Carbo vegetabilis. In another embodiment, the composition further comprises Ignatia, homeopathic ipecac. In another embodiment, the composition further comprises Nux vomica. In another embodiment, the composition further comprises Zingiber officinale.

In another embodiment, the composition of the present invention is in the form of a chewing gum product. In another embodiment, chewing gum compositions contemplated by the present invention comprise all types of sugar and sugarless chewing gums and chewing gum formulations known to those skilled in the art, including regular and bubble gum types. In another embodiment, chewing gum compositions of the invention comprise a chewing gum base, a modifier, a bulking agent or sweetener, and one or more other additives such as, flavoring agents, colorants and antioxidants. In another embodiment, the modifying agents are used to soften, plasticize and/or compatibilize one or more of the components of the gum base and/or of the formulation as a whole.

In another embodiment, the present invention provides a soft, chewable dosage form which is pliable and chewy, yet dissolves quickly in the mouth, has a long shelf life, contains little moisture which improves stability and decreases the tendency for the dosage form to dry out, does not require cooking or heating as part of the manufacturing process. In another embodiment, the dosage form is used as a matrix for dried Cannabis extracts.

In another embodiment, the chewable tablet of the invention comprises a metal salt such as calcium, magnesium, aluminum salt, or any mixture thereof. In another embodiment, the chewable tablet of the invention comprises hydroxyalkyl cellulose. In another embodiment, the chewable tablet of the invention comprises low viscosity hydroxyalkyl cellulose. In another embodiment, the chewable tablet of the invention comprises high viscosity hydroxyalkyl cellulose.

In another embodiment, the chewable tablet of the invention comprises various additives. In another embodiment, the chewable tablet of the invention comprises sweeteners. In another embodiment, the chewable tablet of the invention comprises acidic ingredients. In another embodiment, the chewable tablet of the invention comprises taste correctives. In another embodiment, the chewable tablet of the invention comprises polymeric compounds. In another embodiment, the chewable tablet of the invention comprises essential oils.

In another embodiment, the chewable tablet of the invention is a soft tablet. In another embodiment, the chewable tablet of the invention is made in a state of soft candy. In another embodiment, the chewable tablet of the invention is made in a state of jelly.

In another embodiment, the chewable tablet of the invention comprises a core comprising the vitamins of the invention. In another embodiment, the chewable tablet of the invention comprises an outer layer wrapping the core which is made up of chewable base such as a gum, a soft candy or a caramel.

In another embodiment, the compositions of the present invention may be provided in any suitable food of a solid, semi-solid or liquid form.

The preparation of pharmaceutical compositions that contain a dried Cannabis extract, for example by mixing, granulating, or tablet-forming processes, is well understood in the art. The dried Cannabis extracts are often mixed with excipients that are pharmaceutically acceptable and compatible with the active ingredient. For oral administration, the active ingredients of compositions of the present invention are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions.

In another embodiment, additional methods of administering the dried Cannabis extracts, or compound(s) isolated therefrom, of the invention comprise injectable dosage forms. In another embodiment, the injectable is administered intraperitonealy. In another embodiment, the injectable is administered intramuscularly. In another embodiment, the injectable is administered intradermally. In another embodiment, the injectable is administered intravenously. Each possibility represents a separate embodiment of the present invention.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, additional methods of administering the dried Cannabis extracts of the invention comprise dispersions, suspensions or emulsions. In another embodiment, the dispersion, suspension or emulsion is administered orally. In another embodiment, the solution is administered by infusion. In another embodiment, the solution is a solution for inhalation. Each possibility represents a separate embodiment of the present invention.

In another embodiment, the pharmaceutical composition is administered as a suppository, for example a rectal suppository or a urethral suppository. In another embodiment, the pharmaceutical composition is administered by subcutaneous implantation of a pellet. In another embodiment, the pellet provides for controlled release of active compound agent over a period of time. Each possibility represents a separate embodiment of the present invention.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs. Each possibility represents a separate embodiment of the present invention.

In another embodiment, parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs. Each possibility represents a separate embodiment of the present invention.

In another embodiment, the pharmaceutical compositions provided herein are controlled-release compositions, i.e. compositions in which the active compounds are released over a period of time after administration. Controlled- or sustained-release compositions include formulation in lipophilic depots (e.g. fatty acids, waxes, oils). In another embodiment, the composition is an immediate-release composition, i.e. a composition in which all the active compound is released immediately after administration. Each possibility represents a separate embodiment of the present invention.

In another embodiment, the pharmaceutical composition is delivered in a controlled release system. In another embodiment, the agents are administered using intravenous infusion, an implantable osmotic pump, a transdermal patch, liposomes, or other modes of administration. In another embodiment, a pump is used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials are used; e.g. in microspheres in or an implant. In yet another embodiment, a controlled release system is placed in proximity to the therapeutic target, thus requiring only a fraction of the systemic dose (see, e.g., Goodson, in Medical Applications of Controlled Release, supra, vol. 2, pp. 115-138 (1984); and Langer R, Science 249: 1527-1533 (1990). Each possibility represents a separate embodiment of the present invention.

The compositions also include, in another embodiment, incorporation of the active materials into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance. Each possibility represents a separate embodiment of the present invention.

Also included in the present invention are particulate compositions coated with polymers (e.g. poloxamers or poloxamines) and the compound coupled to antibodies directed against tissue-specific receptors, ligands or antigens or coupled to ligands of tissue-specific receptors. Each possibility represents a separate embodiment of the present invention.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications also increase, in another embodiment, the compound's solubility in aqueous solution, eliminate aggregation, enhance the physical and chemical stability of the compound, and greatly reduce the immunogenicity and reactivity of the compound. In another embodiment, the desired in vivo biological activity is achieved by the administration of such polymer-compound abducts less frequently or in lower doses than with the unmodified compound. Each possibility represents a separate embodiment of the present invention.

The compositions of the present invention may comprise one or more additional components may further include an additional component selected from the group consisting of an anti-static agent, a buffering agent, a bulking agent, a chelating agent, a colorant, a diluent, a dye, an emollient, a fragrance, an occlusive agent, a pH-adjusting agent, a preservative, and a vitamin

The compositions of the present invention may comprise one or more additional active agents, selected from the group consisting of active herbal extracts, analgesics, anti-allergic agents, anti-aging agents, anti-bacterials, antibiotic agents, anticancer agents, antidandruff agents, antidepressants, anti-dermatitis agents, anti-edemics, antihistamines, anti-helminths, anti-hyperkeratolyte agents, anti-inflammatory agents, anti-irritants, anti-microbials, anti-mycotics, anti-proliferative agents, antioxidants, anti-wrinkle agents, anti-pruritics, antiseptic agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, keratolytic agents, lactams, metals, metal oxides, mitocides, neuropeptides, non-steroidal anti-inflammatory agents, oxidizing agents, photodynamic therapy agents, retinoids, sanatives, scabicides, self-tanning agents, skin whitening agents, vasoconstrictors, vasodilators, vitamins, vitamin D derivatives and wound healing agents.

According to some embodiments, the composition may comprise one or more anti-oxidants/radical scavengers. The anti-oxidant/radical scavenger may be selected from butylated hydroxy benzoic acids and their salts, coenzyme Q10, coenzyme A, gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

In one embodiment, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to inducing remission. In another embodiment, “treating” refers to slowing the progression of a disease.

The references cited herein teach many principles that are applicable to the present invention. Therefore the full contents of these publications are incorporated by reference herein where appropriate for teachings of additional or alternative details, features and/or technical background.

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

### REFERENCES

Anampa, J., D. Makower and J. A. Sparano (2015). “Progress in adjuvant chemotherapy for breast cancer: an overview.” BMC Med 13: 195.
DeVita, V. T., S. Hellman and S. A. Rosenberg (2005). Cancer, principles & practice of oncology. Philadelphia, Pa., Lippincott Williams & Wilkins.
Hassan, M. S., J. Ansari, D. Spooner and S. A. Hussain (2010). “Chemotherapy for breast cancer (Review).” Oncol Rep 24(5): 1121-1131.
Thompson, A. M. and S. L. Moulder-Thompson (2012). “Neoadjuvant treatment of breast cancer.” Ann Oncol 23 Suppl 10: x231-236.

1. A method for treating a mammalian disease, the method comprising:
a) combining at least one marijuana cultivar and at least one hemp cultivar to form at least one hybrid line;
b) extracting at least one compound from said at least one hybrid line to form an extract; and
c) administering at least one of said at least one compound and said extract in at least one of in vivo and in vitro to treat, slow or cure said mammalian disease.

2. A method according to claim 1, wherein said at least one hybrid line is selected from the group consisting of a marijuana/marijuana hybrid line, hemp/hemp hybrid line and hemp/marijuana hybrid line.

3. A method according to claim 2, wherein said at least one hybrid line is selected from the group consisting of hybrid number CD10, 4, 10, 18, 20, 24, 28, 30, 33, 40, 41 and 81.

4. A method according to claim 1, wherein said extracting step comprises extracting flowers of said at least one hybrid line.

5. A method according to claim 4, wherein said extracting step comprises extracting flowers in at least one organic solvent.

6. A method according to claim 5, wherein said extracting step is performed at a temperature in the range of 15 to 60° C. and at a pressure in a range of −0.5 to +1.5 bar.

7. A method according to claim 1, wherein said mammalian disease is a proliferative mammalian disease.

8. A method according to claim 7, wherein said proliferative mammalian disease is selected from the group consisting of cancer, breast cancer, neuroblastoma, Ewing sarcoma, Atypical Teratoid Rhabdoid Tumor (ATRT), a cell-cycle deregulation disease, a metastatic disease and combinations thereof.

9. A method according to claim 1, wherein said at least one compound is provided in a concentration in a range of 0.0001-0.05 μg/μl, 0.001-0.05 μg/μl, 0.001-0.005 μg/μl, 0.003-0.03 μg/μl or 0.007-0.015 μg/μl.

10. A method according to claim 1, wherein said at least one compound is provided in a solvent extract and said solvent extract exhibits general anti-cancer effects, effective against a broad range of cancers.

11. A method according to claim 10, wherein said extract is effective against chemo-resistant breast cancer cells and is suitable to overcome chemo-resistance.

12. A method according to claim 10, wherein said extract potentiates effects of cytotoxic chemotherapy and is an effective and safe adjuvant modality.

13. A method according to claim 10, wherein said solvent extract is at least 2-20, 3-15, 4-12, 5-10 or 6-9 times as effective as at least one of THC and CBD, administered at the same concentration in treating said disease.

14. An organic extract of at least one hybrid line, said at least one hybrid line formed from combining at least one of:
a) at least one marijuana or hemp cultivar; and
b) at least one other marijuana or hemp cultivar,
wherein said organic extract comprises at least one compound suitable for treating a mammalian disease.

15. An organic extract according to claim 14, wherein said mammalian disease is a proliferative mammalian disease, selected from the group consisting of cancer, breast cancer, neuroblastoma, Ewing sarcoma, Atypical Teratoid Rhabdoid Tumor (ATRT), a cell-cycle deregulation disease, a metastatic disease and combinations thereof.

16. An organic extract according to claim 15, wherein said extract is effective against chemo-resistant breast cancer cells and is suitable to overcome chemo-resistance.

17. An organic extract according to claim 15, wherein said extract potentiates effects of cytotoxic chemotherapy and is an effective and safe adjuvant modality.

18. An organic extract according to claim 15, wherein said organic extract is at least 2-20, 3-15, 4-12, 5-10 or 6-9 times as effective as at least one of THC and CBD, administered at the same concentration in treating said disease.

19. A combination therapy, isolated from an organic extract of at least one hybrid line, said at least one hybrid line formed from combining at least one of:
a) at least one marijuana or hemp cultivar; and
b) at least one other marijuana or hemp cultivar; and
wherein said organic extract comprises a plurality of compounds suitable for treating a mammalian disease.

20. A combination therapy according to claim 19, wherein said mammalian disease is a proliferative mammalian disease, selected from the group consisting of cancer, breast cancer, neuroblastoma, Ewing sarcoma, Atypical Teratoid Rhabdoid Tumor (ATRT), a cell-cycle deregulation disease, a metastatic disease and combinations thereof.
